(19) United States
(12) Patent Application Publication
(10) Pub. No.: US 2024/0000381 A1
(43) Pub. Date: Jan. 4, 2024

(54) SYSTEM, DEVICE AND METHOD FOR AUTOMATED AUSCULTATION

(71) Applicants: Jean Andino, Chandler, AZ (US); Ally Spohn, Lower Burrell, PA (US)

(72) Inventors: Jean Andino, Chandler, AZ (US); Ally Spohn, Lower Burrell, PA (US)

(73) Assignee: Arizona Board of Regents on behalf of Arizona State University, Scottsdale, AZ (US)

(21) Appl. No.: 18/344,503

(22) Filed: Jun. 29, 2023

Related U.S. Application Data
(60) Provisional application No. 63/356,533, filed on Jun. 29, 2022.

(51) Int. Cl. A61B 5/00; A61B 7/00; A61B 7/04

(52) U.S. Cl. CPC A61B 5/6804; A61B 7/003; A61B 5/002; A61B 5/7203; A61B 7/04; A61B 2562/0204; A61B 2562/046

(57) ABSTRACT

An auscultation system comprises an auscultation device comprising a wearable apparatus positionable proximate to a lung, a plurality of audio sensors embedded in the wearable apparatus configured to record lung sounds, wherein the plurality of audio sensors are positioned at locations of the wearable apparatus corresponding to lung auscultation points, and a computing system communicatively connected to the auscultation device, comprising a processor and a non-transitory computer-readable medium with instructions stored thereon, which when executed by a processor, perform steps comprising, establishing a background noise spectrum, recording a lung sound spectrum via the auscultation device for a period of time, calculating a background subtracted lung sound spectrum by subtracting the background noise spectrum from the measured lung sound spectrum, analyzing the background subtracted lung sound spectrum to produce an auscultation result, and providing the auscultation result to a practitioner to assist in providing a diagnosis.

### CROSS-REFERENCE TO RELATED APPLICATIONS

This application claims priority to U.S. provisional application No. 63/356,533 filed on Jun. 29, 2022, incorporated herein by reference in its entirety.

### BACKGROUND OF THE INVENTION

Doctor visits, particularly for individuals that are either older or immunocompromised can be a challenging endeavor, made even more challenging by an international pandemic. Often times a doctor's visit includes only a few minutes of a discussion, a quick listen to the chest as the person breathes in order to characterize lung and heart sounds, and, depending on what is learned, a plan for a follow-up in 3 to 6 months. Typically a visit with the doctor is 15 to 30 minutes at most, but the preparation and travel time to get to the doctor's office can be 2 to 4 hours, and potentially longer if the patient lives in a remote location. Access to a doctor can often times depend on transportation, which may not be reliable for the most vulnerable populations of society. While some doctors are capable of carrying out telemedicine visits, one persistent challenge is in having the doctor be able to listen—in real time- to the body sounds of the patient.

Furthermore, although it is known that different respiratory diseases cause different lung sounds, there can be discrepancies in how practitioners classify the different lung sounds, thereby leading to potentially different diagnoses and treatments. Knowing how much and which medicine to administer, without providing too high of a dose can be a challenge without a proper diagnosis, or without some measure of how treatments are impacting air movement. More automated tools that can operate in a patient's home could overcome some of these challenges, thereby improving patient outcomes while simultaneously reducing the burdens on the practitioner and enhancing equity in medical access and treatments.

Thus, there is a need in the art for improved systems and methods to enable remote listening to and provide more consistent interpretations of lung sounds, thereby enabling improved real-time monitoring, improved medical diagnoses, and more efficient remote monitoring of the outcomes of therapeutic treatments.

### SUMMARY OF THE INVENTION

Some embodiments of the invention disclosed herein are set forth below, and any combination of these embodiments (or portions thereof) may be made to define another embodiment.

In one aspect, an auscultation device comprises a wearable apparatus positionable proximate to a lung, a plurality of audio sensors embedded in the wearable apparatus configured to record lung sounds, wherein the plurality of audio sensors are positioned at locations of the wearable apparatus corresponding to lung auscultation points, a microcontroller embedded in the wearable apparatus configured to control operation of the plurality of audio sensors, a transceiver embedded in the wearable apparatus, and a power source embedded in the wearable apparatus.

In one embodiment, the wearable apparatus comprises a vest, a strap, a jacket, or a shirt. In one embodiment, the lung auscultation points include at least one of an apex of a lung, an upper lobe of the lung, a middle lobe of the lung, and a lower lobe of the lung. In one embodiment, the wearable apparatus is configured to accommodate variations in body weight or size. In one embodiment, the wearable apparatus is configured to accommodate male and female patients. In one embodiment, the audio sensor comprises a diaphragm, a chest piece, and a sound meter. In one embodiment, the audio sensor comprises a stethoscope. In one embodiment, the power source comprises a battery. In one embodiment, the plurality of audio sensors include four anterior sensors and eight posterior sensors.

In another aspect, an auscultation system comprises an auscultation device comprising a wearable apparatus positionable proximate to a lung, a plurality of audio sensors embedded in the wearable apparatus configured to record lung sounds, wherein the plurality of audio sensors are positioned at locations of the wearable apparatus corresponding to lung auscultation points, a microcontroller embedded in the wearable apparatus configured to control operation of the plurality of audio sensors, a transceiver embedded in the wearable apparatus, and a power source embedded in the wearable apparatus, and a computing system communicatively connected to the auscultation device, comprising a processor and a non-transitory computer-readable medium with instructions stored thereon, which when executed by a processor, perform steps comprising establishing a background noise spectrum, recording a lung sound spectrum via the auscultation device for a period of time, calculating a background subtracted lung sound spectrum by subtracting the background noise spectrum from the measured lung sound spectrum, analyzing the background subtracted lung sound spectrum to produce an auscultation result, and providing the auscultation result to a practitioner to assist in providing a diagnosis.

In one embodiment, the auscultation device is wirelessly communicatively connected to the computing system via a wireless communication protocol comprising 3G, 4G/LTE, 5G, Bluetooth, Bluetooth Low Energy (BLE), Zigbee, near-field communication (NFC), or infrared. In one embodiment, the system further comprises a network communicatively connected to the auscultation device and computing system. In one embodiment, the auscultation device and computing system are wirelessly communicatively connected to the network via a wireless communication protocol comprising 3G, 4G/LTE, 5G, Bluetooth, Bluetooth Low Energy (BLE), Zigbee, near-field communication (NFC), or infrared.

In another aspect, an auscultation system comprises an auscultation device comprising a wearable apparatus positionable proximate to a lung, a plurality of audio sensors embedded in the wearable apparatus configured to record lung sounds, wherein the plurality of audio sensors are positioned at locations of the wearable apparatus corresponding to lung auscultation points, a microcontroller embedded in the wearable apparatus configured to control operation of the plurality of audio sensors, a transceiver embedded in the wearable apparatus, and a power source embedded in the wearable apparatus, and a computing system communicatively connected to the auscultation device, comprising a processor and a non-transitory computer-readable medium with instructions stored thereon, which when executed by a processor, perform steps comprising establishing a baseline lung sound spectrum via data received from the auscultation device, recording a lung sound spectrum via data received from the auscultation device, and identifying a change in a patient's lung sounds over time via comparing the recorded lung sound spectrum to the baseline lung sound spectrum.

In one embodiment, the auscultation device is wirelessly communicatively connected to the computing system via a wireless communication protocol comprising 3G, 4G/LTE, 5G, Bluetooth, Bluetooth Low Energy (BLE), Zigbee, near-field communication (NFC), or infrared. In one embodiment, the system further comprises a network communicatively connected to the auscultation device and computing system. In one embodiment, the auscultation device and computing system are wirelessly communicatively connected to the network via a wireless communication protocol comprising 3G, 4G/LTE, 5G, Bluetooth, Bluetooth Low Energy (BLE), Zigbee, near-field communication (NFC), or infrared.

In another aspect, an auscultation method comprises providing the auscultation system as described above, establishing a background noise spectrum, placing the auscultation device proximate to a patient's lung, recording a lung sound spectrum via the auscultation device for a period of time, calculating a background subtracted lung sound spectrum by subtracting the background noise spectrum from the measured lung sound spectrum, analyzing the background subtracted lung sound spectrum to produce an auscultation result, and providing the auscultation result to a practitioner to assist in providing a diagnosis.

In one embodiment, the method further comprises monitoring over time the outcome of a therapeutic treatment. In one embodiment, the method further comprises activating a subset of the plurality of audio sensors based on a corresponding breathing period. In one embodiment, the breathing period comprises inhalation or exhalation. In one embodiment, the auscultation result is provided to the practitioner located remotely. In one embodiment, the auscultation result is provided to the practitioner in real time. In one embodiment, the method further comprises providing the background subtracted lung sound spectrum to a machine learning algorithm.

In another aspect, an auscultation method comprises providing the auscultation system as described above, establishing a baseline lung sound spectrum via the auscultation device, recording a lung sound spectrum via the auscultation device, and identifying a change in a patient's lung sounds over time via comparing the recorded lung sound spectrum to the baseline lung sound spectrum.

In one embodiment, the step of recording a lung sound spectrum is performed continuously or periodically. In one embodiment, the method further comprises determining at least one of a treatment effectiveness, a disease state, and a disease progression based on the change in a patient's lung sounds over time, and providing at least one of the treatment effectiveness, the disease state, and the disease progression to a practitioner. In one embodiment, the at least one of the treatment effectiveness, the disease state, and the disease progression is provided to the practitioner located remotely. In one embodiment, the at least one of the treatment effectiveness, the disease state, and the disease progression is provided to the practitioner in real time. In one embodiment, the change in a patient's lung sounds over time is identified via a machine learning algorithm.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a table summarizing continuous adventitious respiratory sounds in accordance with some embodiments.

FIG. 2 is a table summarizing discontinuous adventitious respiratory sounds in accordance with some embodiments.

FIGS. 3A and 3B depict an exemplary auscultation system including an exemplary auscultation device in accordance with some embodiments.

FIG. 4 is a flow-chart depicting an exemplary auscultation method in accordance with some embodiments.

FIG. 5 is a flow-chart depicting an exemplary method for monitoring a patient's change in lung sounds over time in accordance with some embodiments.

FIG. 6 is a table summarizing characteristics of respiratory system conduits in accordance with some embodiments.

FIG. 7 depicts a schematic of an exemplary experimental setup where the MFC and MFM are the mass flow controller and mass flow meter, respectively.

FIG. 8 is a plot depicting exemplary experimental sound intensity level as a function of cross-sectional area and airflow rate.

FIG. 9 is a table summarizing fitted exemplary experimental sound intensity levels as a function of cross-sectional area (A) and airflow rate (Q).

FIG. 10 is an exemplary experimental flow rate residual plot for the silicone straw with a 0.5 cm diameter.

FIG. 11 is an exemplary experimental flow rate residual plot for the silicone straw with a 0.75 cm diameter.

FIG. 12 is an exemplary experimental flow rate residual plot for the silicone straw with a 1.0 cm diameter.

FIG. 13 is an exemplary experimental flow rate line fit plot comparing the recorded and fitted sound intensity levels.

FIG. 14 is an exemplary experimental cross-sectional area line plot comparing the recorded and fitted sound intensity levels.

FIG. 15 is a plot showing exemplary experimental recorded and fitted sound intensity levels as a function of flow rate for the silicone straw with a 0.50 cm diameter.

FIG. 16 is a plot showing exemplary experimental recorded and fitted sound intensity levels as a function of flow rate for the silicone straw with a 0.75 cm diameter.

FIG. 17 is a plot showing exemplary experimental recorded and fitted sound intensity levels as a function of flow rate for the silicone straw with a 1.0 cm diameter.

FIG. 18 depicts a schematic of an exemplary experimental setup.

FIG. 19 is a plot showing exemplary experimental actual sound intensity levels vs. predicted sound intensity levels where the diagonal line is the model where the data points are the recorded sound intensity levels.

FIG. 20 is a plot showing exemplary experimental residuals comparing the observed sound intensity levels with the predicted model.

FIG. 21 is a plot showing exemplary experimental sound intensity level (dB) as a function of flow rate (L/min) for the cross-sectional area of 0.18 cm². The recorded sound intensity values are shown with markers and the model is plotted as a dotted line. Error bars represent the standard deviation from the average values.

FIG. 22 is a plot showing exemplary experimental sound intensity level (dB) as a function of flow rate (L/min) for the cross-sectional area of 0.73 cm². The recorded sound intensity values are shown with markers and the model is plotted as a dotted line. Error bars represent the standard deviation from the average values.

FIG. 23 is a plot showing exemplary experimental sound intensity level (dB) as a function of flow rate (L/min) for the cross-sectional area of 1.27 cm². The recorded sound intensity values are shown with markers and the model is plotted as a dotted line. Error bars represent the standard deviation from the average values.

FIG. 24 is a plot showing exemplary experimental sound intensity level (dB) as a function of flow rate (L/min) of all the straws tested. The recorded sound intensity values for each individual cross-sectional area are plotted with different color markers. The models for each cross-sectional area are plotted with their respective dotted lines. Error bars represent the standard deviation from the average values.

FIG. 25 is a plot showing exemplary experimental sound intensity level (dB) as a function of cross-sectional area (cm²). The recorded sound intensity values for each individual flow rate are plotted with different color markers. The models for each flow rate are plotted with their respective dotted lines. Error bars represent standard deviation from the average values.

FIG. 26 depicts an exemplary computing environment in which aspects of the invention may be practiced in accordance with some embodiments.

### DETAILED DESCRIPTION OF THE INVENTION

It is to be understood that the figures and descriptions of the present invention have been simplified to illustrate elements that are relevant for a clearer comprehension of the present invention, while eliminating, for the purpose of clarity, many other elements found in systems, devices and methods of automated auscultation. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein are systems, devices and methods for automated auscultation.

Within every human body is a network of organs and tissues that allow one to breathe. This intertwined network is known as the respiratory system, comprising of airways, including the mouth, nose, sinuses, pharynx, trachea, bronchial tubes, and lungs, and additional components, such as the diagram, ribs, alveoli, bronchioles, cilia, and blood vessels. Together, these organs and tissues allow for a human body to inhale air from its surrounding environment, then absorb the oxygen and move it throughout the body while removing carbon dioxide waste at the same time₁.

By understanding the functions of the different components of the respiratory system, the flow of air can be analyzed. Pulmonary ventilation, which is the process of breathing, is the movement of air in and out of the lungs 2. When air flows through the respiratory system, it first enters the human body through the mouth or nose, then travels through the pharynx, known as the upper respiratory tract. As air progresses to the lower respiratory tract, it flows into the trachea, where it begins to flow towards the lungs through the bronchial tubes and their small branches, known as bronchioles. The bronchioles finally transport air into the lungs through the alveoli, which are air sacs present in the lungs, where the oxygen and carbon dioxide exchange occurs₁.

Both inspiration and expiration depend on pressure differences between the lungs and the atmosphere, supported Boyle's Law which describes the relationship between pressure and volume as being inversely proportional, as represented by the following equation₂:

P₁V₁=P₂V₂ (1)

Ventilation depends on atmospheric pressure (Pₐₜₘ), intra-alveolar pressure (Pₐₗᵥ), and intrapleural pressure (Pᵢₚ). Atmospheric pressure, equal to 1 atm, is “the amount of force that is exerted by gases in the air surrounding any given surface”₂. Intra-alveolar pressure, or intrapulmonary pressure, is “the pressure of the air within the alveoli, which changes during the different phases of breathing”₂. Intrapleural pressure is “the pressure of the air within the pleural cavity, between the visceral and parietal pleurae” and changes during the different breathing phases₂. The intra-alveolar pressure equalizes with the atmospheric pressure during ventilation, whereas the intrapleural pressure is always lower. Transpulmonary pressure is “the difference between the intrapleural and intra-alveolar pressures, and it determines the size of the lungs”₂.

Before inspiration occurs, the diaphragm and external intercostal muscles contract to allow for the thoracic cavity volume to increase, resulting in a decrease in pressure within the lungs and a pressure gradient driving airflow, as supported by Boyle's Law. During inspiration, air flows down its gradient from the atmosphere to the lungs because atmospheric pressure is the greatest, followed by intra-alveolar and intrapleural pressures. However, during expiration, the pressure in the lungs is greater than atmospheric pressure. Thus, after gas exchange occurs, air flows down its pressure gradient out of the lungs into the atmosphere₂.

Respiratory volume refers to the volume of air moved in and out of the lungs during the respiratory cycle. Tidal volume (TV) is the amount of air that enters the lungs during quiet breathing, which occurs at rest₃. The expiratory reserve volume (ERV) is the amount of air that can be forcefully exhaled, whereas inspiratory reserve volume (IRV) is the amount of air that can be forcefully inhaled by the lungs. Residual volume (RV) is the amount of air that remains in the lungs after exhalation. The total lung capacity is the sum of tidal volume, expiratory reserve volume, inspiratory reserve volume, and residual volume. However, the vital capacity (VC) is the amount of air that can be moved in and out of the lungs, so it is the sum of tidal volume, expiratory reserve volume, and inspiratory reserve volume. The respiratory rate is the number of breaths that occur each minute where this air moves in and out of the lungs, allowing for gas exchange. When a patient has a respiratory disease, their respiratory rate is likely to increase the amount of air entering the lungs₂.

Due to the complexity of the respiratory system, several respiratory diseases can affect the human body and a person's ability to breathe. These conditions may occur at any point throughout the respiratory system. When diagnosing these respiratory conditions and diseases, physicians use a process known as auscultation, which involves performing a physical exam and listening to airflow within a patient's chest using a stethoscope₄. A stethoscope comprises a tube connected to a round metal piece, known as the chest piece, with a surrounding diaphragm, or plastic piece. When a physician places a stethoscope on a patient's chest, the sound of the airflow will cause the diaphragm to vibrate, and the sound eventually travels through the connected tube to the physician's ears. By listening to a patient's chest, a physician can identify whether the respiratory sound is normal or abnormal, then diagnose them with a respiratory condition or disease based on the sound and their symptoms.

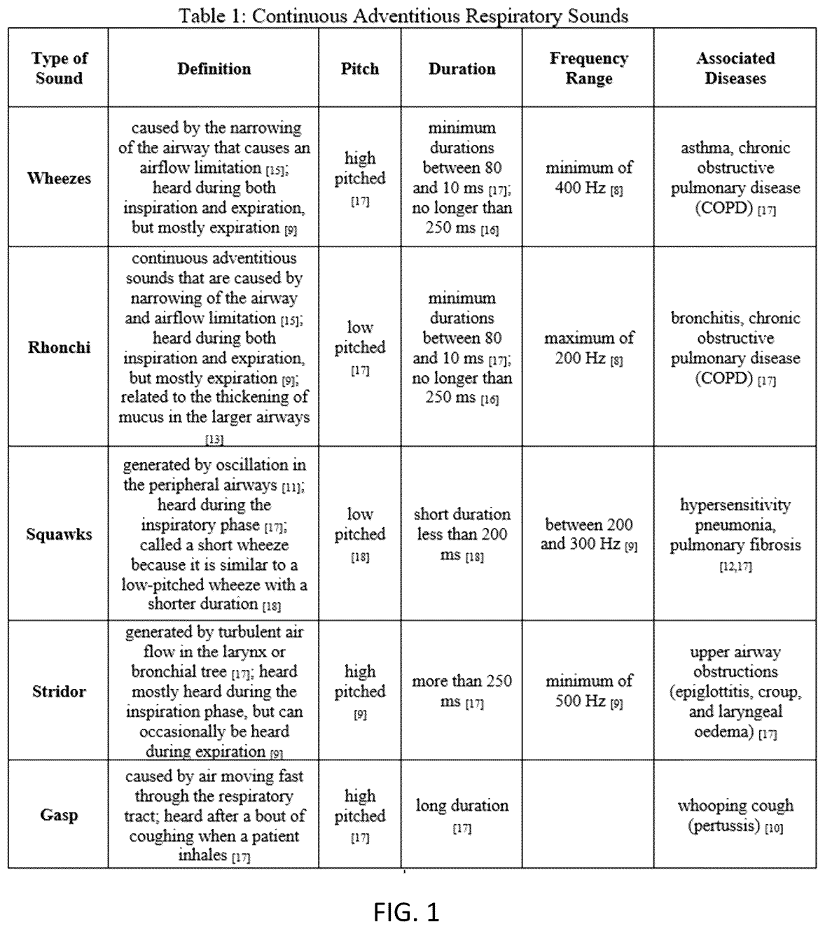

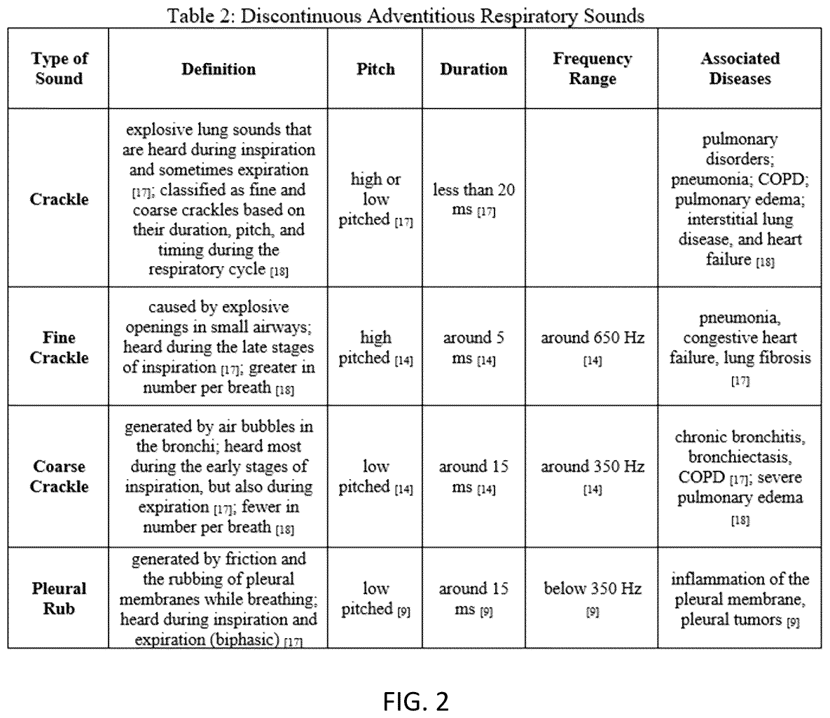

Breath sounds are normal lung sounds that are heard during auscultation. They are classified as either bronchial, vesicular, or bronchovesicular and have different properties which depend on the location where the physician is performing the auscultation. Bronchial sounds are heard over the tracheobronchial tree and are loud, harsh, and high-pitched sounds heard during expiration, whereas vesicular sounds are heard over the lung parenchyma and are soft and low-pitched sounds heard during inspiration when air flows through an open airway_{6, 7}. Bronchovesicular sounds are mid-range pitch and intensity sounds heard over the upper anterior chest during inspiration and expiration. Adventitious sounds are “heard in addition to the expected breath sounds” and can be either continuous or discontinuous, as seen in the following tables of FIG. 1 and FIG. 2₆.

The continuous adventitious respiratory sounds include wheezes, rhonchi, squawks, stridor, and gasps. Wheezes are caused by narrowing of an airway that results in an airflow limitation and are heard during inspiration and expiration, but mostly expiration_{9, 15}. They have a high pitch with a minimum frequency of 400 Hz and are associated with asthma and chronic obstructive pulmonary diseases_{8, 17}. Rhonchi are caused by large airway narrowing and airflow limitation due to secretions or the thickening of mucus in the airway_{9, 13, 15}. They are heard during inspiration and expiration with a low pitch and maximum frequency of 200 Hz₈. These sounds are associated with bronchitis and chronic obstructive pulmonary disease₁₇. Squawks are generated by oscillation in the peripheral airways and heard during inspiration_{11,18}. They are low-pitched wheezes with a frequency between 200 and 300 Hz that are typically associated with pneumonia and pulmonary fibrosis_{8, 17}. Stridors are generated by upper airway obstructions and the turbulent airflow that they cause. These sounds are high-pitched with a minimum frequency of 500 Hz and mostly heard during inspiration, but sometimes during expiration_{9,17}. Finally, gasps are caused by fast-moving air in the respiratory tract and heard during inhalation when coughing. They are high pitched and heard in patients with pertussis, or whooping cough_{10,17}.

The discontinuous adventitious sounds are crackles and pleural rubs. Crackles are explosive lung sounds heard during inspiration and sometimes expiration₁₇. They are classified as fine and coarse crackles depending on duration, pitch, and timing during respiration's. Fine crackles are caused by explosive openings and fluid secretions in small airways, such as the bronchioles. They are heard during the late stage of inspiration and are short, high-pitched sounds with a frequency of around 650 Hz and are associated with pneumonia, congestive heart failure, and lung fibrosis_{14,18}. Coarse crackles are generated when air bubbles and secretions are present in the bronchi and heard during the early stages of inspiration₁₇. They are longer, low-pitched sounds with a frequency of around 350 Hz and are associated with chronic bronchitis, bronchiectasis, chronic obstructive pulmonary disease, and severe pulmonary edema_{14,18}. Pleural rubs are generated by friction and rubbing of pleural membranes while breathing and heard during inspiration and expiratior₁₇. They are low pitched with a frequency below 350 Hz and associated with inflammation of the pleural membrane and pleural tumors₉.

Air flow through the trachea-bronchial tree generates breath sounds. This, however, depends on the type of flow. Laminar flow is not responsible for producing breath sound since it is associated with low flow situations and the air flows parallel to the tubes. When there is a higher flow flowing through a larger diameter, this gets classified as turbulent flow. Turbulent flow produces breath sounds due to the flow's random and chaotic nature. As the air molecules collide with one another and along the surfaces of the inner vessels, the flow becomes less laminar and more turbulent.

It is expected that an increase in air flow rate will result in an increase in sound intensity levels. An increase in air flow rate can be due to more forceful inhalation or exhalation, resulting in increased sound levels. In addition, the increase in turbulence from increased flow rights indicates molecules are colliding with each other and along the tube walls. When flow is smaller, it becomes more laminar as there is less of a chance for molecule collisions. However, when considering the respiratory system, the impact of cross-sectional areas on sound intensity levels is complex. It is generally expected that an increase in cross-sectional areas will also yield higher sound intensity levels. Larger cross-sectional areas, like the bronchi, can lead to more turbulent flow. Smaller cross-sectional areas, such as small airways, yield more laminar flow as the air has more of a confined space to travel through. There are more factors to consider when determining the effect of cross-sectional area. When a person has a respiratory illness that can cause inflammation or obstruction in the airways, the resistance to airflow is increased. This yields a higher driving pressure required for air flow. This can disrupt laminar flow, creating turbulent flow which leads to increased sound intensity levels. Although there are more factors to consider, the setup of this experiment is more simplified, therefore an increase in sound intensity levels with increasing air flow rate and cross-sectional area is expected. By improving the understanding of sound intensity level's dependence on cross sectional area and air flow rate, this can be correlated to a monitoring technique that diagnoses respiratory illnesses based on variations in sound level intensities.

As indicated in Tables 1 and 2 of FIGS. 1 and 2, respectively, adventitious respiratory sounds correspond to different respiratory diseases, allowing physicians to diagnose respiratory diseases after performing auscultation. Respiratory diseases may present differently in each patient, but one commonality is the acoustics associated with each of them. Therefore, physicians can diagnose diseases after analyzing the sound, pitch, duration, and frequency of respiratory sounds 3. The systems and methods disclosed herein are configured to monitor airflow within the human respiratory system and further configured to perform advancing sensing, acoustic signal processing, and machine learning to enhance the automation of respiratory sounds. This provides for better detection and diagnosing of respiratory diseases, such as asthma, chronic obstructive pulmonary disease (COPD), bronchitis, and pneumonia_{17,18}.

Asthma is a chronic disease caused by the inflammation of the airway and excessive mucus secretions that inhibit air from entering the lungs 2. Symptoms of asthma include coughing, shortness of breath, wheezing, and chest tightness, which may be exacerbated by exercise, stress, respiratory infections, or environmental factors, including dust, pollen, pet dander, weather changes, mold, and smoke 19. When a patient is experiencing an asthma attack, these symptoms worsen and require immediate medical attention due to difficulty breathing and its side effects, such a lack of oxygen, confusion, drowsiness, and rapid pulse. In terms of treatment, there are various short and long-term methods that depend on the severity and frequency of the condition. The most common type of short-term treatment is an inhaler, which contains steroids that reduce airway inflammation, allowing a better flow of oxygen to the lungs₂.

Chronic obstructive pulmonary disease is a chronic inflammatory lung disease that causes obstructed airflow to the lungs. It is typically caused by emphysema and chronic bronchitis. Chronic bronchitis is the long-term inflammation of the bronchial tube lining and is characterized by daily cough and mucus production. Emphysema is a condition in which the alveoli of the lungs are destroyed from exposure to cigarette smoke and other irritating gases and particulate matter. Symptoms of COPD include chronic cough, shortness of breath, wheezing, chest tightness, production of mucus, frequent respiratory infections, and lack of energy. In terms of treatment, bronchodilators, steroids, and lung therapies are used, but most importantly, patients with COPD must quit smoking₂₀.

Bronchitis is the inflammation of the lining of the bronchial tubes. It can be either acute or chronic with acute caused by a viral infection, such as a cold or flu, and chronic caused by long-term exposure to smoke, air pollution, and dust. Symptoms of bronchitis include coughing, shortness of breath, production of mucus, fatigue, slight fever, and chest discomfort. In terms of treatment, most people get better with time in few weeks, but sometimes cough medications, inhalers, and steroids are needed₂₁.

Pneumonia is a viral or bacterial infection that leads to inflammation of air sacs with fluid or pus in one or both lungs. The symptoms of pneumonia include chest pain when breathing or coughing, cough, production of mucus, fatigue, fever, and shortness of breath. These symptoms may range from mild to life-threatening. In terms of treatment, antibiotics, cough medicine, and fever reducers are mostly used₂₂.

Other applications for the disclosed system and method can include quick diagnoses of warfighter health in theater when medical facilities may be far away, and applications where sensing air flow through the use of sound can be applied in industrial settings such as monitoring scaling in pipes, where the differences in sound pitch in pipes with and without scale buildup may result in prompt diagnoses of imminent pipe structural failures.

In some embodiments, the systems and methods disclosed herein involve air transport in the human respiratory system, (including the chemical and physiological impacts of air pollutants and therapeutics for respiratory diseases) and acoustic signal processing (including both training and machine learning) to enhance automation.

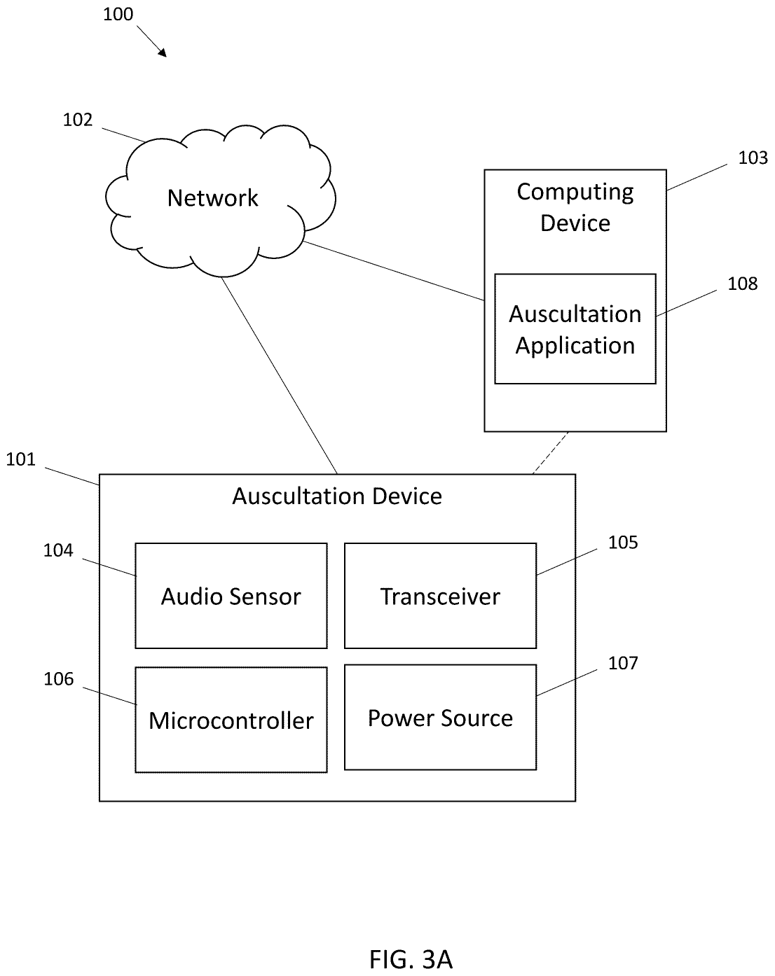

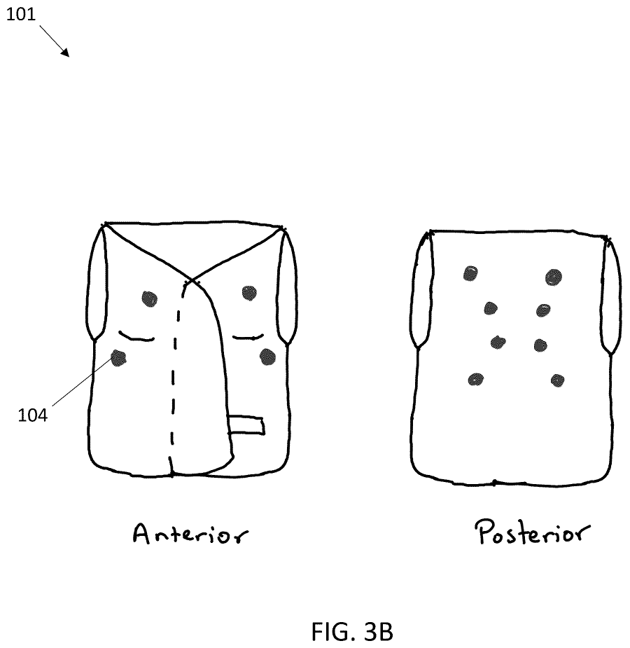

Referring now to FIGS. 3A and 3B, an exemplary auscultation system 100 including an exemplary auscultation device 101 is shown. In some embodiments, the system 100 and device 101 are configured to perform lung auscultation. In some embodiments, the auscultation device 101 comprises a wearable apparatus with a plurality of audio sensors 104 embedded in the wearable apparatus configured to record lung sounds. In some embodiments, the plurality of audio sensors 104 are positioned at locations of the wearable apparatus corresponding to lung auscultation points. In some embodiments, the device further includes a microcontroller 106 embedded in the wearable apparatus configured to control operation of the plurality of audio sensors 104, a transceiver 105 embedded in the wearable apparatus configured to communicatively connect with a computing device 103 and/or a network 102, and/or a power source 107 embedded in the wearable apparatus configured to power the audio sensors 104, transceiver 105, and microcontroller 106. In some embodiments, the power source 107 comprises a battery or other suitable power source.

In some embodiments, the wearable apparatus comprises a vest, a strap, a jacket, a shirt, or any other suitable wearable apparatus or combination thereof. In some embodiments, the lung auscultation points include at least one of an apex of a lung, an upper lobe of the lung, a middle lobe of the lung, and a lower lobe of the lung. In some embodiments, the wearable apparatus is configured to accommodate variations in body weight, size, and to accommodate male and female patients.

In some embodiments, the audio sensor 104 comprises a diaphragm, a chest piece, transmitter, and a sound meter. In some embodiments, the audio sensor 104 comprises a stethoscope. In some embodiments, the plurality of audio sensors 104 include four anterior sensors and eight posterior sensors. In some embodiments, the device 100 includes sound dampening padding proximate to the audio sensors 104.

In some embodiments, the device 101 can be configured to activate a subset of the plurality of audio sensors 104 at a point corresponding to a breathing period, such as inhalation or exhalation, to enable proper recording of lung sounds. In some embodiments, the system 100 and device 101 are configured to be used individually by a person without the aid of another individual, and without the need for knowledge of where to place sensors to perform proper auscultation. In some embodiments, the system 100 includes a trainable diagnostic component to aid a practitioner in their diagnoses. In some embodiments, the diagnostic component can rely on acoustic signal processing, lung sound training, and/or machine learning to improve initial diagnoses in real time. In some embodiments, the auscultation application 108 includes the trainable diagnostic component.

The system 100 can further include a computing system 103 communicatively connected to the auscultation device 101.

In some embodiments, the computing system 103 can include a processor and a non-transitory computer-readable medium with instructions stored thereon, which when executed by a processor, perform steps of an auscultation method comprising establishing a background noise spectrum, recording a lung sound spectrum via the auscultation device 101 for a period of time, calculating a background subtracted lung sound spectrum by subtracting the background noise spectrum from the measured lung sound spectrum, analyzing the background subtracted lung sound spectrum to produce an auscultation result, and providing the auscultation result to a practitioner to assist in providing a diagnosis. In some embodiments, an auscultation application 108 running on the computing device is configured to perform at least a portion of the auscultation method.

In some embodiments, the computing system 103 can include a processor and a non-transitory computer-readable medium with instructions stored thereon, which when executed by a processor, perform steps of method for monitoring a patient's change in lung sounds over time, comprising establishing a baseline lung sound spectrum via data received from the auscultation device 101, recording a lung sound spectrum via data received from the auscultation device 101, and identifying a change in a patient's lung sounds over time via comparing the recorded lung sound spectrum to the baseline lung sound spectrum. In some embodiments, a patient monitoring application, such as auscultation application 108, running on the computing device is configured to perform at least a portion of the method for monitoring a patient's change in lung sounds over time.

In some embodiments, the auscultation device 101 is wirelessly communicatively connected to the computing system 103 via a wireless communication protocol comprising 3G, 4G/LTE, 5G, Bluetooth, Bluetooth Low Energy (BLE), Zigbee, near-field communication (NFC), infrared, or any other suitable wireless communication protocol. In some embodiments, the auscultation device 101 is communicatively connected to the computing system 103 via a wired connection such as ethernet, USB, or any other suitable wired connection.

In some embodiments, the system 100 further comprises a network 102 communicatively connected to the auscultation device 101 and computing system 103. In some embodiments, the auscultation device 101 and computing system 103 are wirelessly communicatively connected to the network 102 via a wireless communication protocol comprising 3G, 4G/LTE, 5G, Bluetooth, Bluetooth Low Energy (BLE), Zigbee, near-field communication (NFC), infrared, or any other suitable wireless communication protocol. In some embodiments, the auscultation device 101 and the computing system 103 are communicatively connected to the network 102 via a wired connection such as ethernet, USB, or any other suitable wired connection. In some embodiments, the auscultation device 101 and the computing system 103 are communicatively connected to the network 102 via any combination of wired or wireless connection.

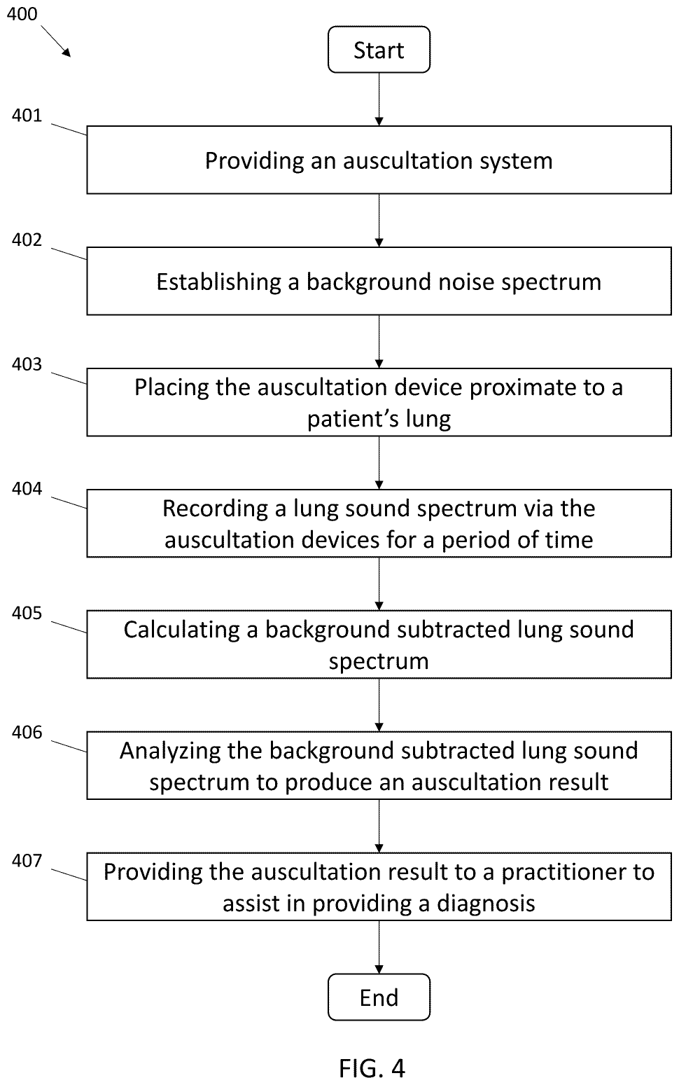

FIG. 4 is a flowchart depicting an exemplary auscultation method 400. The method 400 starts at Operation 401 where an auscultation system 100 is provided. In some embodiments, the auscultation system 100 includes an auscultation device 101 comprising a wearable apparatus, and a plurality of audio sensors 104 embedded in the wearable apparatus configured to record lung sounds. In some embodiments, the plurality of audio sensors 104 are positioned at locations of the wearable apparatus corresponding to lung auscultation points.

At Operation 402 a background noise spectrum is established. In some embodiments, the background noise spectrum is measured prior to placing the auscultation device 101 proximate to a lung. In some embodiments, the background noise spectrum is measured after placing the auscultation device 101 proximate to a lung. In some embodiments, the background noise spectrum is measured fora period of time. In some embodiments, the period of time ranges from 1 second to 1 hour, 10 seconds to 15 minutes, 1 minute to 5 minutes, or about 2 minutes.

At Operation 403 the auscultation device 101 is placed proximate to a patient's lung. In some embodiments, the device 101 is a wearable device and is thus placed proximate to the patient's lung by putting the wearable device on the patient. In some embodiments, the wearable device is a vest, strap, shirt, jacket, or any other suitable wearable device or combination thereof.

At Operation 404 a lung sound spectrum is recorded via the auscultation device 101 for a period of time. In some embodiments, the period of time ranges from 1 second to 1 hour, 10 seconds to 15 minutes, 1 minute to 5 minutes, or about 2 minutes.

At Operation 405 a background subtracted lung sound spectrum is calculated by subtracting the background noise spectrum from the measured lung sound spectrum. Any suitable background subtraction method can be utilized.

At Operation 406 the background subtracted lung sound spectrum is analyzed to produce an auscultation result. In some embodiments, the background subtracted spectrum is analyzed for known patterns that are representative of known diseases. In one example, these patterns can be determined through comparison to the auditory database that is available for lung sounds. In some embodiments, during use of the system 100 to assess the effectiveness of treatments, the new auscultation sound profile can be compared to a saved baseline auscultation sound profile to assess the effectiveness of the treatment process. In some embodiments, regular calibration of the system with baseline auscultation sounds is obtained by the user. Whether comparing the new sound profile pattern to the training sound pattern or to baseline sound patterns, mathematical analyses can be conducted to determine differences. Particularly when comparing profiles from the same individual (e.g. the “baseline” versus “treatment” sound profiles, statistical analyses can be employed to determine the similarities of the sound profiles to each other. The extent of difference or similarity of the sound profiles can be used to determine whether additional treatment is or is not needed. In some embodiments, a threshold similarity value is calculated when comparing the baseline and treatment sound profiles, and if the similarity threshold is less than a set value, a recommendation to start treatment is provided. In some embodiments, the similarity threshold is about 20% to 80% similar, about 20% similar, about 30% similar, about 40% similar, about 50% similar, about 60% similar, about 70% similar, about 80% similar, or any other suitable threshold. In some embodiments, the similarity threshold can be determined via a clinical trial.

The method 400 ends at Operation 407, where the auscultation result is provided to a practitioner to assist in providing a diagnosis. The result can be provided via a report, a suggested diagnosis, a diagnosis confidence level list, or any other suitable method. The result can further be provided via a display on a device such as a smartphone, computer, or other suitable smart or computing device 103, or can be provided via a paper report or similar.

In some embodiments, an auscultation application 108 running on a computing device 103 is configured to perform the background subtraction, auscultation analysis, and or display the results. In some embodiments, an auscultation application 108 running on a computing device 103 is configured to provide breathing point instructions to a patient, such as instructing the patient to breathe in and breath out for specific time durations and frequency. In some embodiments, the method 400 further comprises providing the background subtracted lung sound spectrum to a machine learning algorithm.

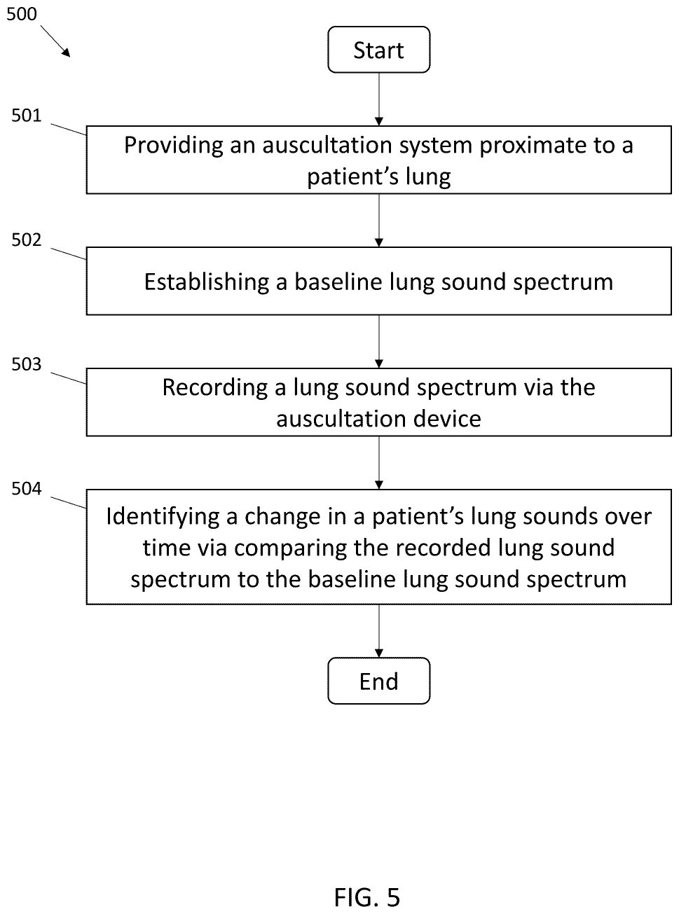

FIG. 5 is a flow-chart depicting a method 500 for monitoring a patient's change in lung sounds over time. The method 500 starts at Operation 501 where an auscultation system 100 is provided. In some embodiments, the auscultation system 100 includes an auscultation device 101 comprising a wearable apparatus, and a plurality of audio sensors 104 embedded in the wearable apparatus configured to record lung sounds. In some embodiments, the plurality of audio sensors 104 are positioned at locations of the wearable apparatus corresponding to lung auscultation points.

At Operation 502, a baseline lung sound spectrum is established. In some embodiments, the baseline lung sound spectrum is measured for a period of time. In some embodiments, the period of time ranges from 1 second to 1 hour, 10 seconds to 15 minutes, 1 minute to 5 minutes, or about 2 minutes.

At Operation 503, a lung sound spectrum is recorded via the auscultation device 101 for a period of time. In some embodiments, the period of time ranges from 1 second to 1 hour, seconds to 15 minutes, 1 minute to 5 minutes, or about 2 minutes. In some embodiments, the lung sound spectrum is recorded continuously and/or periodically.

The method 500 ends at Operation 504, where a change in a patient's lung sounds over time is identified via comparing the recorded lung sound spectrum to the baseline lung sound spectrum. In some embodiments, the change in a patient's lung sounds over time is identified via a machine learning algorithm.

In some embodiments, the method 500 further includes establishing a background noise spectrum, and calculating a background subtracted lung sound spectrum and background subtracted baseline lung sound spectrum by subtracting the background noise spectrum from each respective lung sound spectrum.

In some embodiments, the method 500 further includes determining a treatment effectiveness, a disease state, and/or a disease progression based on the change in a patient's lung sounds over time, and providing the treatment effectiveness, the disease state, and/or the disease progression to a practitioner. In some embodiments, the at least one of the treatment effectiveness, the disease state, and the disease progression is provided to the practitioner located remotely and/or in real time.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore specifically point out exemplary embodiments of the present invention and are not to be construed as limiting in any way the remainder of the disclosure.

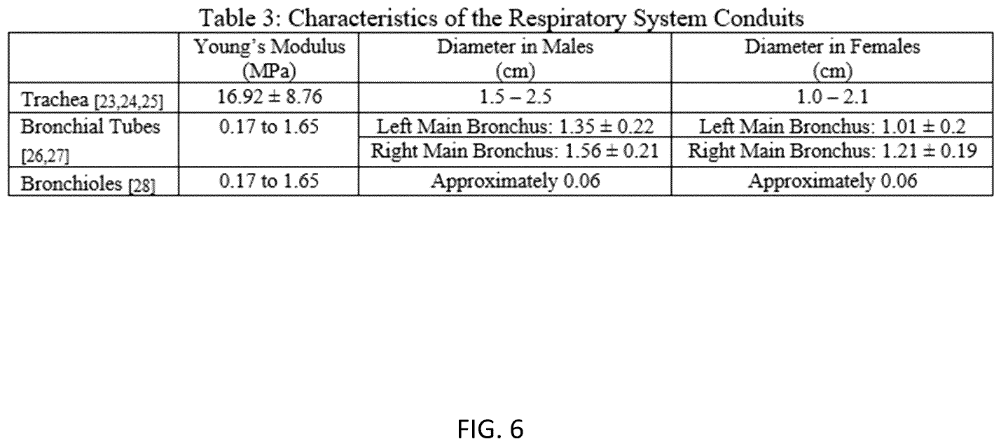

To understand and analyze airflow within the respiratory system, the respiratory system conduits first needed to be simulated. These conduits include the trachea, bronchial tubes, and bronchioles, as these are primary conduits in which air flows through before entering the lung, with their characteristics included in Table 3 of FIG. 6₂.

Due to their similar elasticity, silicone straws with a Young's Modulus of approximately 1.0 MPa were chosen to simulate the conduits of the respiratory system, specifically the bronchial tubes_{29, 30}. Three silicone straws were used with diameters of 0.5 cm, 0.75 cm, and 1.0 cm since these diameters were relatively similar those of the trachea and bronchial tubes. However, it was desired to 3D print the trachea, bronchial tubes, bronchioles, and lungs using silicone, but this was challenging with time constraints.

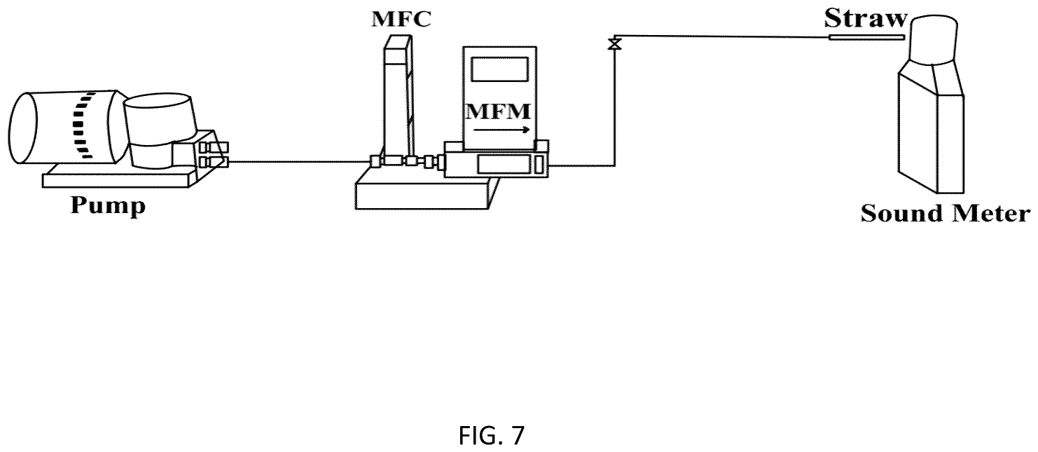

The system used was designed to determine whether there is a mathematical relationship between airflow rate, cross-sectional area of the conduits, and sound. Once analyzed, this relationship was applied to the respiratory system to determine how the narrowing of an airway affects the sound intensity levels of airflow. It was hypothesized that as flow rate and cross-sectional area increased, sound intensity level increased as a result. To determine whether this hypothesis was supported, three silicone straws with different diameters and three different airflow rates were evaluated. The schematic of the experimental setup that was used during the experiment is presented in FIG. 7.

The exhaust port of the pump was used to produce airflow. Teflon tubing with a 0.25-in outer diameter was used to connect the pump to the remainder of the sampling train. A Swagelok “T” was connected to a mass flow controller and a mass flow meter. By controlling the airflow rate that passed through and exited the mass flow controller, the remaining flow that eventually passed through the silicone straw could be varied. The flow that entered the silicone straw was measured using the mass flow meter that was positioned before the silicone straw. After flowing through the mass flow controller and meter, the air continued to flow through a series of connected 0.25-in tubes until it eventually entered a valve that was opened or closed. When opened, air continued to flow through tubing until it entered the attached straw being evaluated. The air flowed through the straw, which ended 0.5 cm from the sound meter, which detected and recorded the sound of airflow exiting the straw. The sound meter used was an ennoLogic eS528L Sound Level Meter. During the experiment, the same setup was used for the 0.5 and 0.75 cm straws. However, for the straw with the 1.0 cm diameter, a 0.5-in tube was attached to the 0.25-in tube before the straw was attached to account for the difference in diameter, resulting in a slightly longer setup.

While measuring the sound of airflow, it was important to ensure that the fittings were airtight to guarantee that no airflow was lost to the surroundings and the intended flow rate was reaching the straw and sound meter. As previously mentioned, three diameters, 0.5 cm, 0.75 cm, and 1.0 cm, and three flow rates, 1.25 L/min, 1.72 L/min, and 2.13 L/min, were analyzed. The diameter of the cylindrical silicone straws was used to determine their cross-sectional areas using the equation:

$$A = π * {( \frac{d}{2} )}^{2} \qquad ( 2 )$$

Three trials were conducted at each flow rate and cross-sectional area to allow error propagation. Flow rates were chosen based on measurements of airflow within the human body. In a healthy adult, tidal volume is approximately 0.5 L, and their breathing rate is 12 breaths/min. Therefore, using the following equation the minute ventilation, which is the volume of air entering the lungs each minute, was calculated to be 6 L/min 3.

Minute Ventillation=Tidal Volume×Breathing Rate (3)

The airflow rate of 6 L/min was the desired flow rate to be tested during the experiment, but the pump in the laboratory did not produce a high enough flow rate for this value to be tested. As a result, lower flow rates with equal differences between them were tested. Despite being lower than the desired 6 L/min, these values will allow for proper analyses to determine whether there is a relationship between flow rate, cross-sectional area, and sound₃.

Before conducting each trial, the background sound was recorded for two minutes. This background sound was a combination of the sound in the room where the experiment was performed and the sound of the pump which produced the airflow. While the background sound was being recorded, the valve was closed to stop air from flowing through the tube. However, once the value was opened, the sound of air flow was recorded for the following two minutes. This process was repeated twice, allowing for three sound level measurements for each flow rate and cross-sectional area. The average background and flow rate sound levels were compared using a T-Test with Two Sample Means in Microsoft Excel. For all trials, the two-sided p-values were far less than the 5% level of significance, indicating that the results were statistically significant. This demonstrates that the changes in sound level were due to the changes in flow rates within the silicone straws, as opposed to chance. However, when comparing the background and flow rate sound levels, the data needed to be corrected using the following equation:

$${L}_{flow} = 10 * log [ {10}^{\frac{{L}_{total}}{10}} - {10}^{\frac{{L}_{background}}{10}} ] \qquad ( 4 )$$

Where L_{flow} is the actual sound intensity level in decibels (dB) generated by the air flow, Lₜₒₜₐₗ is the sound intensity level of the background and air flow, and L_{background} is the sound intensity level of the background noise.

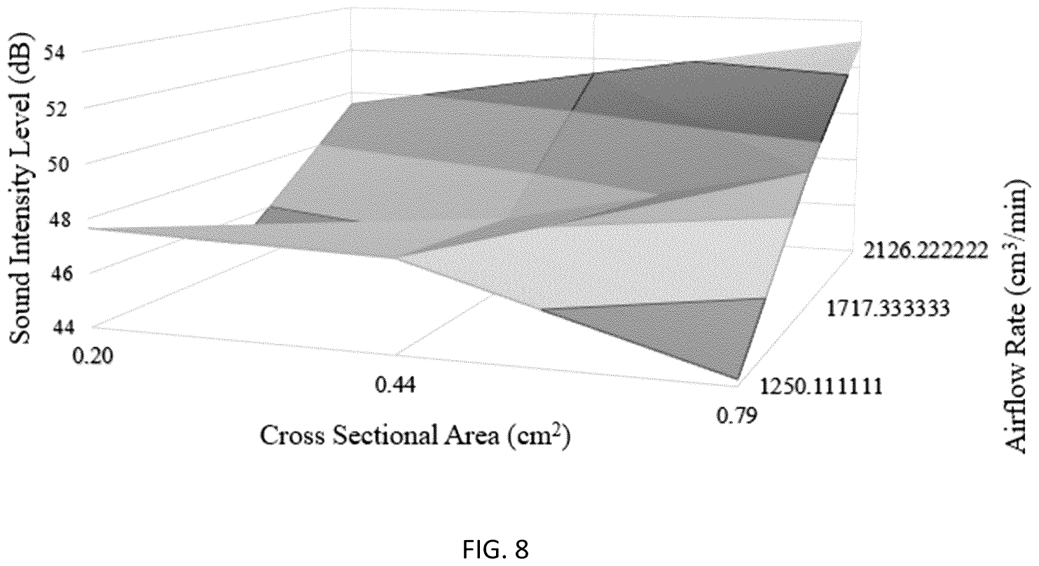

By converting the recorded sound to sound intensity level, the background sound was removed from the flow rate sound, allowing for only the flow rate sound to be analyzed. FIG. 8 includes the sound intensity levels as a function of airflow rate and cross-sectional area.

While conducting the experiment, it was desired to test the same three flow rates at all three diameters. It was challenging to do because the pump did not produce the high desired flow rate despite the accuracy of the mass flow controller used. The average airflow rates analyzed were 1250.11, 1726.67, and 2126.22 cm³/min with the silicone straw diameters being 0.5, 0.75, and 1.0 cm.

As seen in FIG. 8, when the diameter remained constant and the flow rate increased, the sound intensity level experienced a steady increase. However, for the 0.5 cm and cm diameters, the lowest sound intensity level was at an average flow rate of 1717.33 cm³/min. The sound intensity level for the 1.0 cm silicone straw increased steadily as the flow rate increased. Furthermore, when the airflow rate remained constant and the cross-sectional area increased, the sound intensity level changed as expected. The results were inconclusive because for the 0.5 cm silicone straw, the sound intensity level experienced a decrease from 47.66, 47.38, and 44.24 dB as the flow rate increased from 1250.11, 1726.67, and 2126.22 cm3/min. However, for the 0.75 and 1.0 cm silicone straws, the sound intensity level increased from 44.92, 46.72, to 49.21 dB and 49.44, 51.31, to 53.11 dB, respectively, as the airflow rate increased. This discrepancy is likely due to inconsistencies in the background sound since the sound intensity level was calculated by subtracting the background sound from the total sound, including the flow rate and background sound.

Nonlinear regression was utilized to fit the flow rate, cross-sectional area, and sound data based on the following equation:

L_{flow}_{_{fitted}}=a+b*log(Q)+c*log(A) (5)

Where L_{flow_fitted} is the fitted sound intensity level in dB, Q is the flow rate in cm³/min, A is the cross-sectional area in cm³, and a, b, and c are the fitting constants. After performing nonlinear regression using the regression analysis in Microsoft Excel, it was determined the relationship is as follows:

L_{flow}_{_{fitted}}=−13.35+19.43*log(Q)+2.56*log(A) (6)

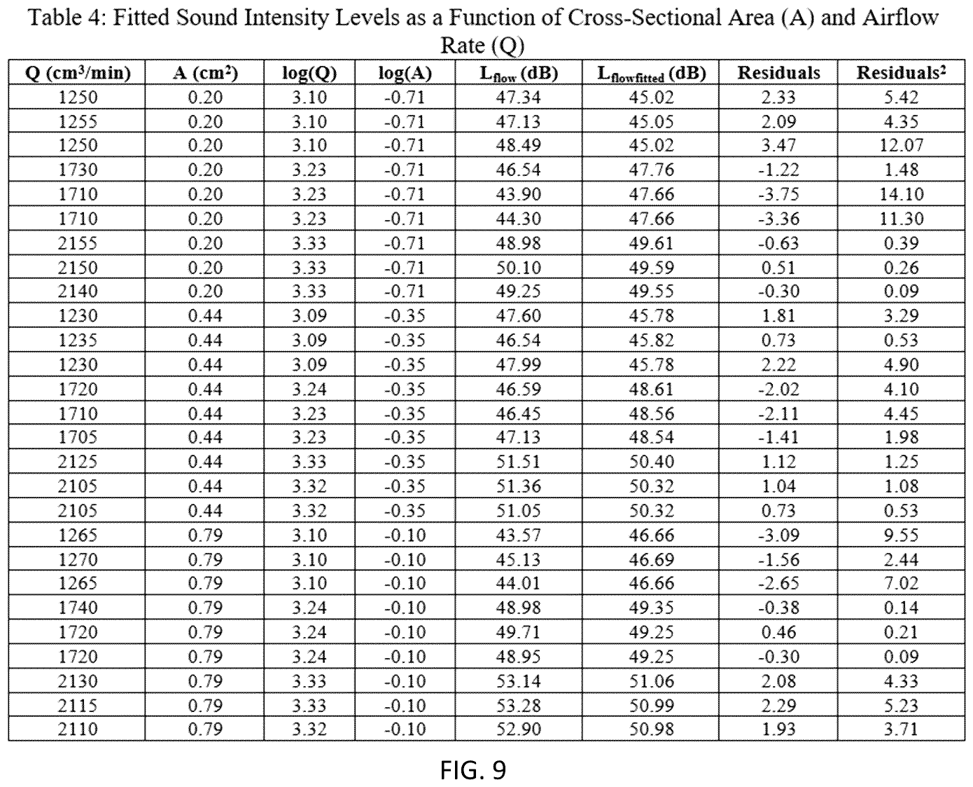

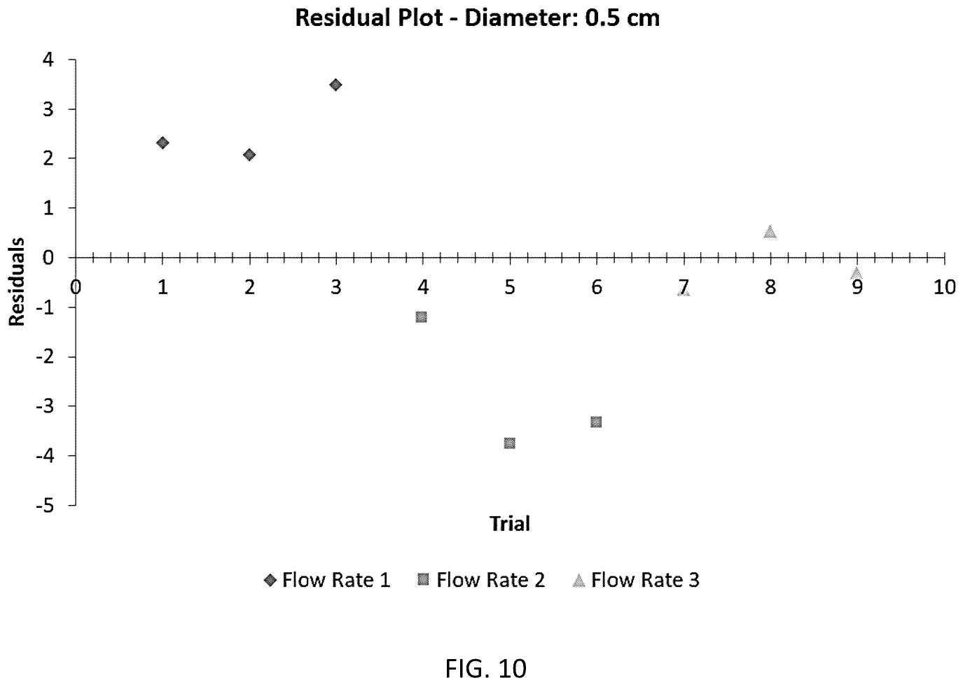

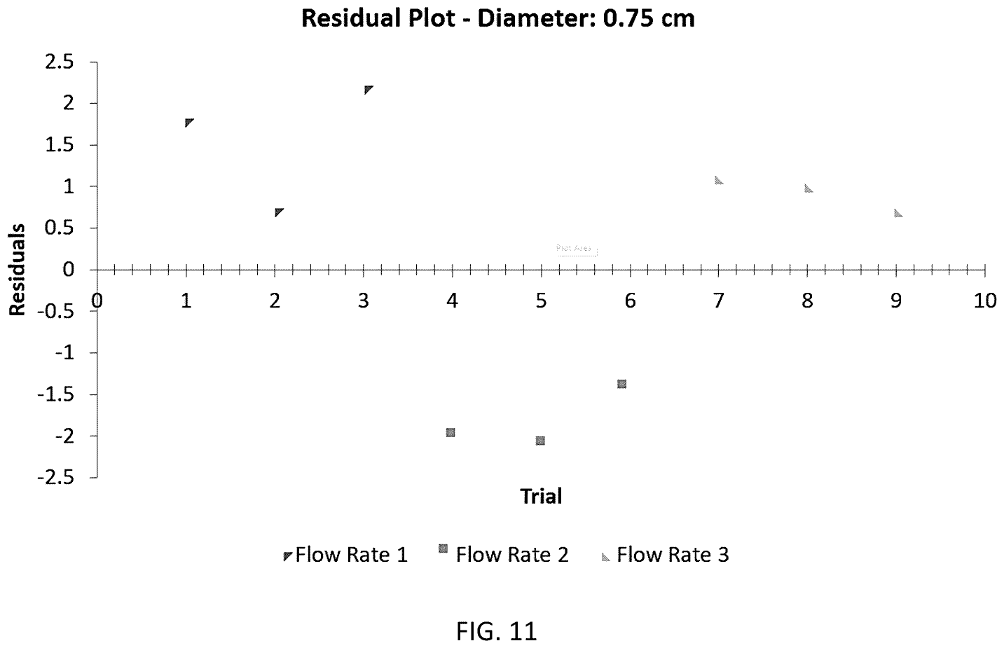

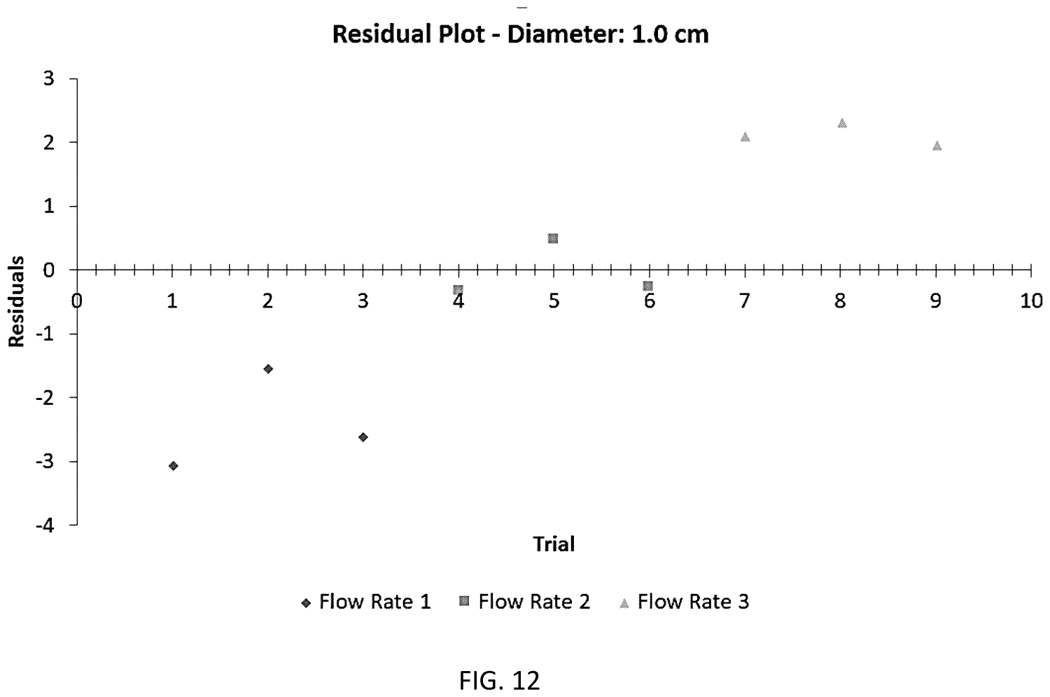

The fitted sound intensity levels determined from equation 6 are presented in Table 4 of FIG. 9. As seen in Table 4 of FIG. 9, the recorded sound intensity levels are compared to the fitted levels. The sound values all tend to increase in response to an increase in flow rate and cross-sectional, as expected by the hypothesis. However, that is not necessarily the case for the recorded sound intensity levels for the straws with 0.5 and 0.75 cm. The residual plots for each straw, demonstrating the differences between the recorded and fitted sound intensity levels, are presented in FIGS. 10, 11, and 12.

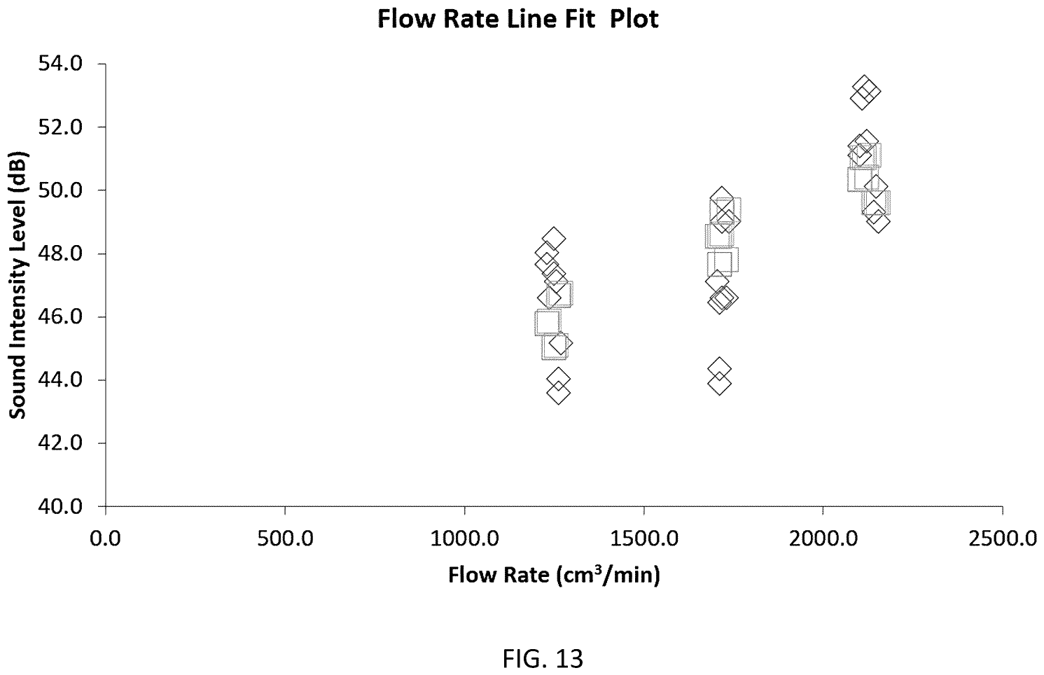

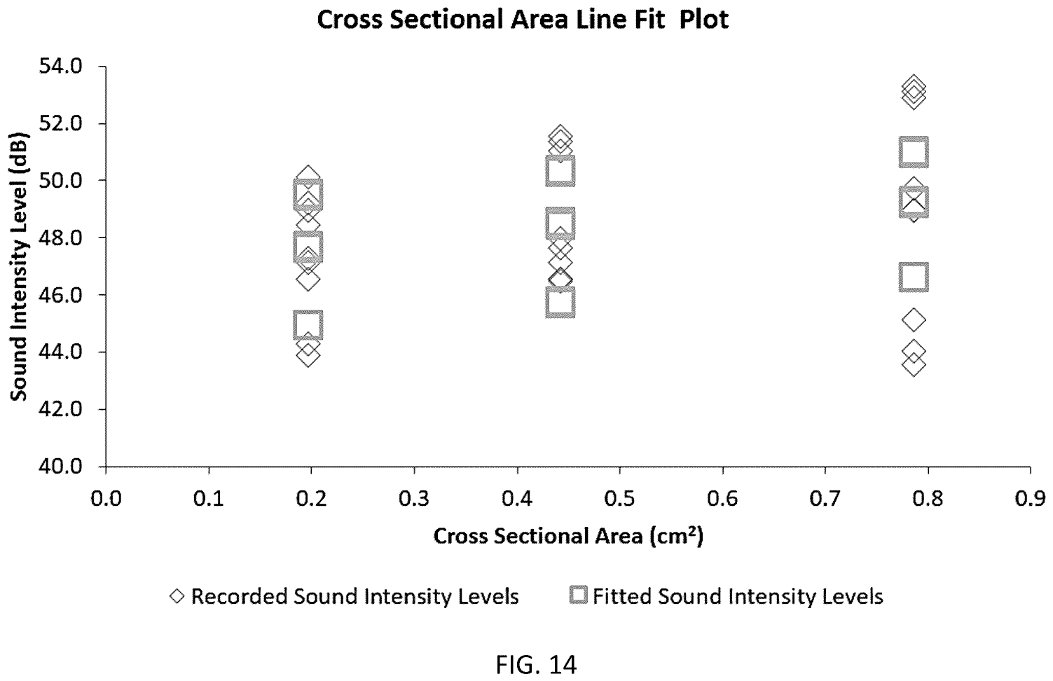

The residuals are the differences between the fitted and recorded sound intensity levels and determine whether the relationship is a good fit for the experimental data. It is expected to have residuals that slightly vary from 0 with overall average and sum of residuals squared of 0. Based on FIGS. 10, 11, and 12, the residuals did fluctuate near 0 as expected for all three straws with the sum of residuals being equal to 104.30, which was far higher than the desired value of 0. As previously mentioned, these discrepancies and larger residuals were due to human error and a slightly louder, inconsistent background sound that lowered the recorded sound intensity levels after the background sound was subtracted from the total recorded sound. To further determine whether this relationship is a good fit, the line fit plots are included in FIGS. 13 and 14.

Based on FIGS. 13 and 14, it can be concluded that the predicted relationship is a good fit for the data values since the recorded and fitted sound intensity levels align for the most part. To further prove that the relationship indicated above is a good fit, a t-test was performed to compare the recorded sound intensity level with the fitted sound levels, which produced a Significance F value of 0.00027. When using a 95% confidence interval, it was determined that there is a dependent relationship between the sound intensity level, airflow rate, and cross-sectional area because the results were statistically significant since 0.00027 is less than 0.05. Before conducting the experiment, it was hypothesized that there is a dependent relationship between flow rate, cross-sectional area, and sound intensity levels. Preliminary data suggest that results support this hypothesis with the changes in sound intensity levels being attributed to changes in flow rate and cross-sectional area based on the Significance F value. However, additional data are required in order to improve the fit.

When evaluating the coefficients associated with the air flowrate and cross-sectional area parameters individually, the coefficient that was associated with the flowrate had a standard error of 4.23 and a p-value of 0.00012, far less than the level of significance of 0.05. These statistics suggest that sound intensity levels are dependent on airflow rate. However, for cross-sectional area, the standard error was 1.62 and the p-value was 0.13, which is not statistically significant because it is greater than 0.05. This result suggests that more information is needed in order to better represent the relationship between the cross-sectional area term of the modelled fit equation (i.e., equation 6) and the sound intensity level.

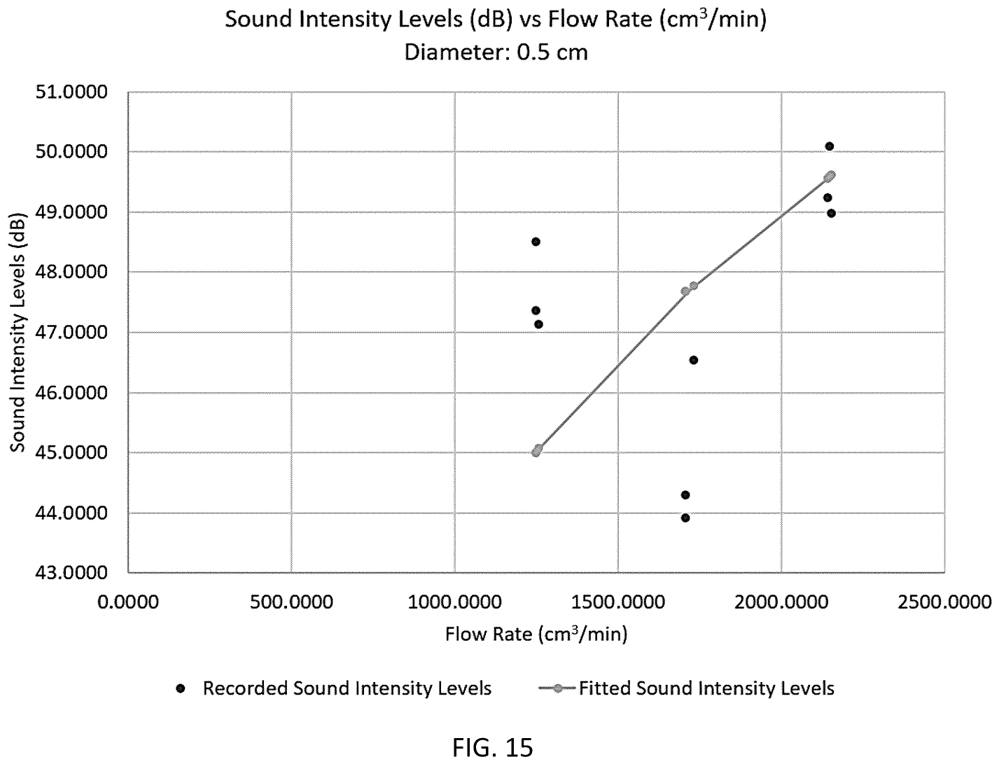

In FIGS. 15, 16, and 17, the recorded sound intensity levels are compared to the fitted sound intensity levels as functions of airflow rate and cross-sectional area.

As shown above in FIG. 15, the sound intensity level decreased from an average of 47.66 to 44.92 dB when the flow increased from 1251.67 to 1716.67 cm³/min, which is a discrepancy from the fitted sound intensity levels presented. However, the sound intensity level greatly increased from an average of 44.92 to 49.44 dB when the flow rate increased from 1716.67 to 2148.33 cm³/min. Since the recorded sound level increased as the airflow rate increased, the decrease present in the sound intensity level at 1251.67 cm³/min was due to a slightly louder background sound at 53.44 dB, whereas the background sounds at 1251.67 and 2148.33 cm³/min were 52.36 and 53.26 dB, respectively.

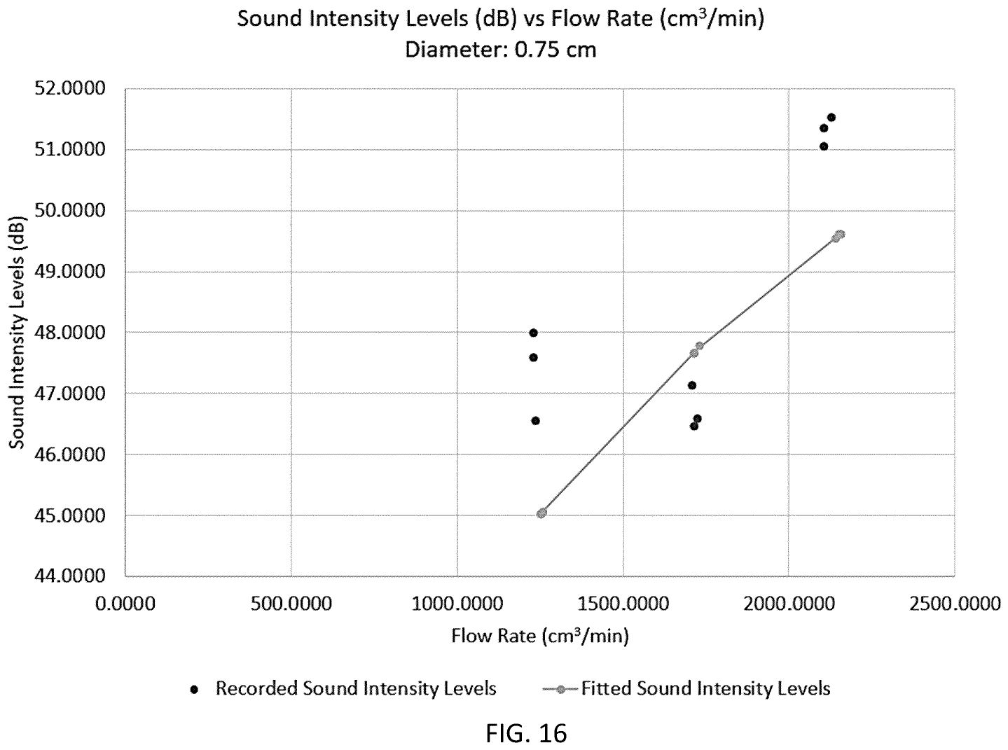

FIG. 16 also shows that the sound intensity level slightly decreased from 47.38 to 46.72 dB when the flow increased from 1231.67 to 1711.67 cm³/min for the 0.75 cm straw. However, the sound intensity level greatly increased from 46.72 to 51.31 dB when the flow rate increased from 1711.67 to 2111.67 cm³/min, as expected. This was because the background sound was louder than expected at 52.90 dB and its difference between the total recorded sound was not as great as the other flow rates, 1231.67 and 2111.67 cm³/min and their respective background sounds of 52.24 and 53.69 dB.

The results indicate that even though the total recorded sound increased as flow rate increased, the increase was not entirely due to flow rate. Once the background sound was subtracted from the total sound, there was a decrease in the average sound intensity level present at the middle flow rate tested for both straws. Since it was predicted that the sound intensity would increase, this decrease can be attributed to sounds present in the background that the sound meter detected (e.g. human movement), or the equipment was not properly set up. Before and after conducting each trial, the equipment was checked thoroughly to ensure that everything was airtight and the straw was 5 mm from the sound meter, but there could have been slight movement once air began to flow through the equipment, thereby affecting the distance and results. Furthermore, the pump produced a constant sound, in addition to the valve controlling whether air flow was passing through the straw. When the valve was closed, it produced a sound that was detected by the sound recording device. However, that sound from the valve was not present when the valve was opened, allowing airflow through the straw. Ultimately, this affected the sound intensity levels determined by the recorded background and total sound.

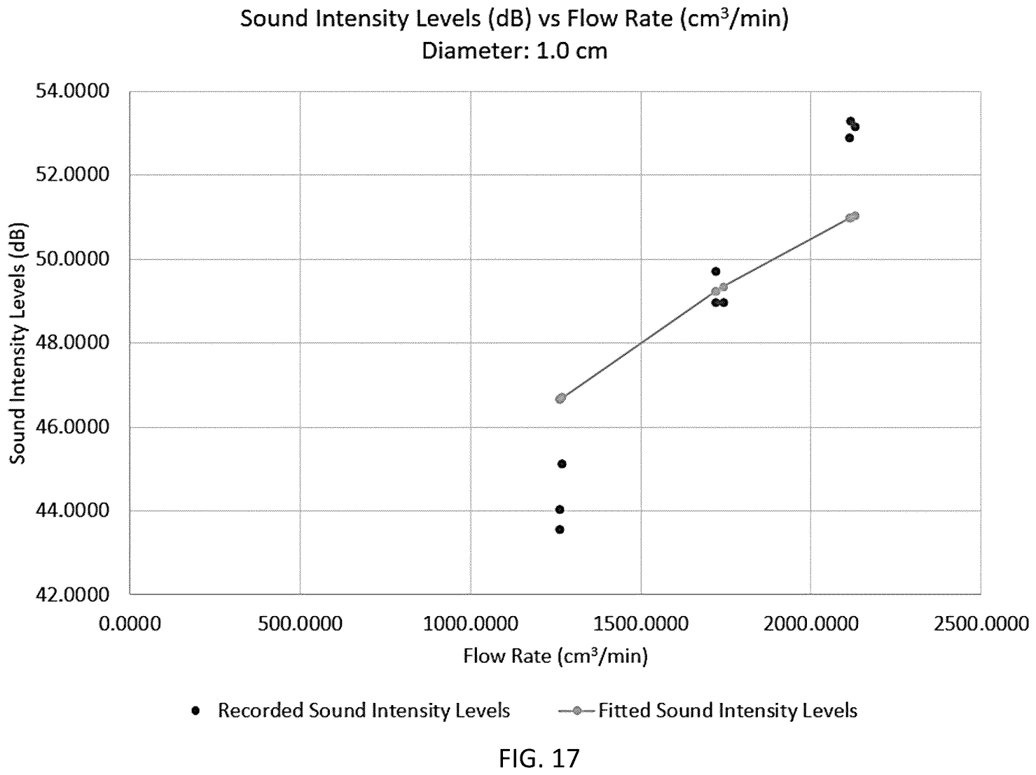

As seen in FIG. 17, the sound intensity level increased from 44.24, 49.21, to 53.11 dB when the flow increased from 1266.67, 1726.67, to 2118.33 cm³/min, as expected. Amongst all the diameters evaluated, the silicone straw with the 1.0 cm has the best fitting set of data to the predicted relationship. Based on the statistical analysis and figures presented above comparing the recorded sound intensity levels to the fitted levels, it was concluded that all the data sets were good fits to the predicted relationship between sound intensity levels, airflow rate, and cross-sectional area.

As cross-sectional area and airflow rate increase, the resulting sound intensity level produced from the air flowing through the silicone straw tends to increase. This indicates that louder sounds can be heard with greater flow within a vessel, such as the trachea, bronchial tubes, or bronchioles. Therefore, there is potential to utilize this mathematical relationship between sound intensity levels, cross-sectional area, and airflow rate to advance sensing, acoustic signal processing, and machine learning to enhance the automation of respiratory sounds. In doing so, the sound of airflow can be measured and compared to normal sound intensity levels to determine whether a patient is experiencing narrowing of their airway or decreased airflow_{4,5}. However, to allow for greater connection of this mathematical relationship to the respiratory system in the future, air flow will need to be simulated in 3D printed respiratory conduits with a higher airflow rate between 6 and 15 L/min, as this is the rate in which air flows through the respiratory tract in an adult₃. Altogether, this relationship can be used as an indicator of whether a patient has a respiratory disease caused by the narrowing of an airway and can aid in a patient's diagnosis.

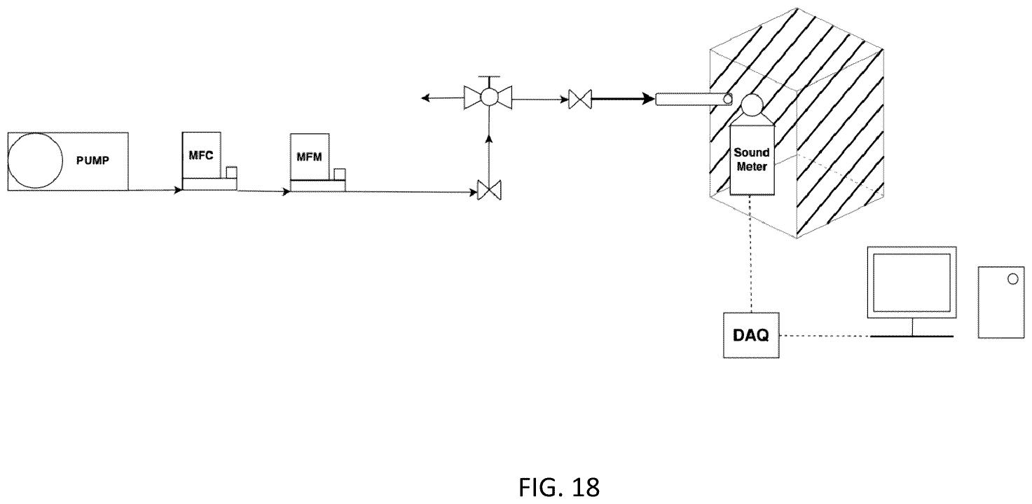

FIGS. 18 through 25 detail additional experimental details and results. In order to accurately represent the characteristics of the respiratory system, it was critical to select the appropriate materials and dimensions that imitate the properties of the human airways. Silicone straws were chosen to represent the respiratory vessels as they have similar elasticity to vessels within the respiratory system. This is also under the assumption that the vessels are perfectly cylindrical in shape and smooth. To account for the variety of vessel diameters within the respiratory system, various silicone straws were tested. The inner diameters of the silicone straws tested had inner diameters of 0.19 cm, 0.38 cm, and 0.5 cm, which correspond to the cross-sectional areas of 0.18 cm², 0.73 cm², and 1.27 cm², respectively.

The equipment utilized in this study comprised a Gast Pump DOA-P704-AA for air flow rate facilitation. Teflon tubing with an inner diameter of 0.19 in was utilized to transfer the air flow rate from throughout the equipment. Precise control and readings of the air flow rates tested was accomplished using an Aalborg GFC37 mass flow controller and Aalborg GFM37 mass flow meter. A Swagelok 3-way ball valve was used to redirect airflow in order to measure background noise and the air flow noise emanating from the straw. The tubing leading to the straw was connected to another tube that had a diameter of 0.31 in. The sound level intensities were recorded using the ennoLogic eS528L sound meter. A sound deadening system was placed around the sound meter to also try and eliminate background noise. This was a cardboard box with padding around the inside. Lastly, a data acquisition system (DAQ) was employed by connecting the sound meter to a desktop computer. The EnnoLogic Smart Logger 2 software was used to retrieve the data for further analysis and interpretation.

To minimize the impact of various background noises measured by the meter, the pump was placed outside the room of testing. To further reduce any potential sources of noise, a box lined with sound-deadening foam was placed around the meter. The goal of this was to ensure that the measured sound levels primarily reflected the noise generated by the air flow through the straws with varying diameters.

The tubing connected to a pump was attached to the mass flow controller and mass flow meter on the other end. More tubing was attached and connected to the ball valve. At the end of the Teflon tubing, the silicone straw was attached to the tubing. The sound meter was placed 0.5 away from the end of the straw, ensuring consistent measurements across various flow rates and straw diameters. The schematic of this setup is shown in FIG. 18.

The experimental procedure involved first turning on the mass flow controller and meter and allowing them to warm up for 25 minutes to reach full accuracy. The apparatus, including the sound meter and sound-deadening system, was then prepared for data collection. The pump was turned on and the air flow rate was adjusted to the desired value using the mass flow controller, while simultaneously monitoring the values on the mass flow meter. The valve was positioned to the left to prevent airflow from entering the tubing and straw, and instead, exit through a separate outlet. This was done to record the background noise for a duration of two minutes. Following that, the valve was redirected to the right and the air flow went through the tubing and out of the silicone straw. The sound recordings were taken for another two minutes. The data recorded during both phases were then analyzed to determine the impact of the straw and airflow on the noise levels produced.

The flow rates studied ranged from 6 to 11 L/min, increasing by increments of 1 L/min. These values were chosen as these represent the minute ventilation at which humans breath, as discussed previously. These six different flow rates were tested with each of the silicone straws.

The data collected with the sound meter was retrieved using the Ennologic Smart Logger 2 Software. The equation used for data analysis is shown in Equation 4. The units of these parameters are in decibels. This equation allows the background noise to be disregarded from the sound intensity levels so the only data analyzed is the noise coming from the straw.

To evaluate the effectiveness of the sound deadening system in reducing background noise, a comparative test was carried out. This involved measuring the background noise levels both with and without the sound deadening system. The sound-deadening system was effective in eliminating background noises by 3.1 dB, from 53.9 dB without the sound-deadening system to 50.8 dB with the sound deadening system. The addition of the system successfully reduced unwanted noise interference during data collection, which allowed for a more controlled research environment.

All the data was fitted to a model using Polymath. Raw data was inputted along with a skeleton model of y=c+b*log(A)+a*log(Q). The model along with the appropriate constant parameters are shown in Equation 7. The R-squared adjusted value was used to assess the goodness of fit. The value was 0.87, indicating a significant relationship between the independent variables and the dependent variable.

L_{Air Flow}=53.88+8.83*log(A)+16.41*log(Q) (7)

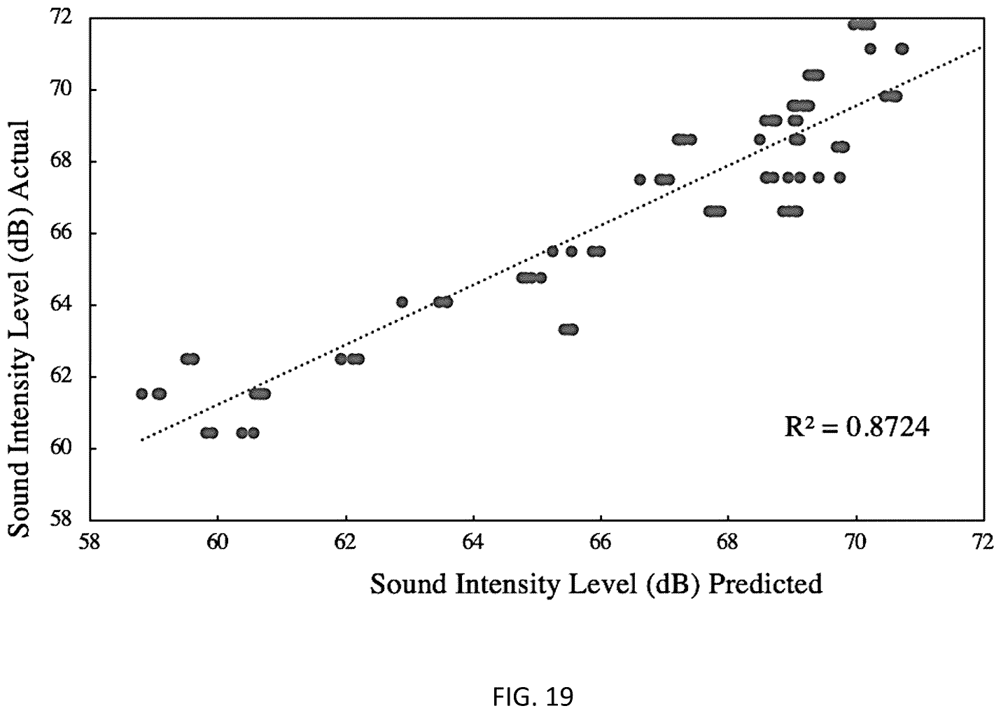

The actual predicted plot, as shown in FIG. 19, provides a visual representation of the relationship between the recorded sound intensity levels and the predicted values from the model. It serves as an assessment of how well the model's predictions align with the observed data. Deviations from the diagonal line indicate discrepancies between the predicted and actual values. Ideally, the points should cluster around the perfect fit line, indicating a good fit. In this plot, there are no significant points of variation, suggesting a favorable and accurate fit between the model and the actual data.

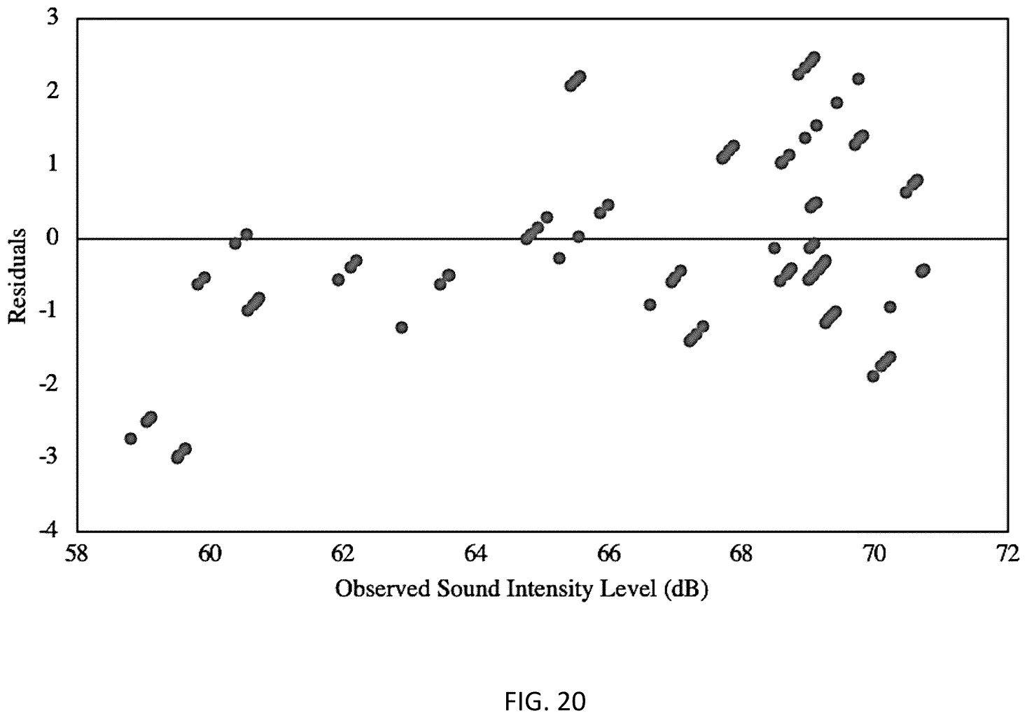

Another plot examined was the residuals plot, as shown in FIG. 20. Residuals are important in assessing the accuracy of a statistical model. This is done by measuring the discrepancies between the observed data and the model's predictions. Ideally, perfect residuals should show no pattern around the data points plotted around zero. Since there is no pattern shown in this plot, this indicates there is no bias within the model and that the model's assumptions are valid. This also indicates that the model's predictions are a good representation of the recorded data.

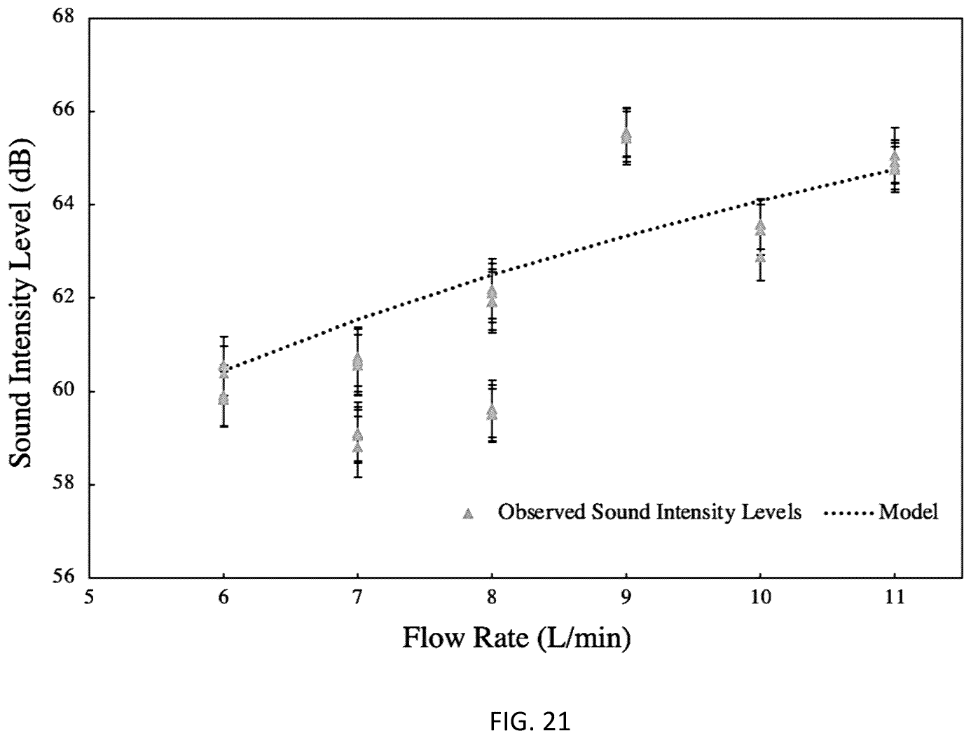

As mentioned previously, the sound intensity levels are expected to increase with increasing flow rate. FIG. 21 plots the sound intensity levels against flow for the smallest straw size. When looking at the plot visually, the trend of the observed sound levels generally increases but there are some variations when comparing the model fit. When averaging the data points at each flow rate, the sound level at 9 L/min produced the largest deviation of about 2 dB. This variation likely could have been due to some error when performing the experiment.

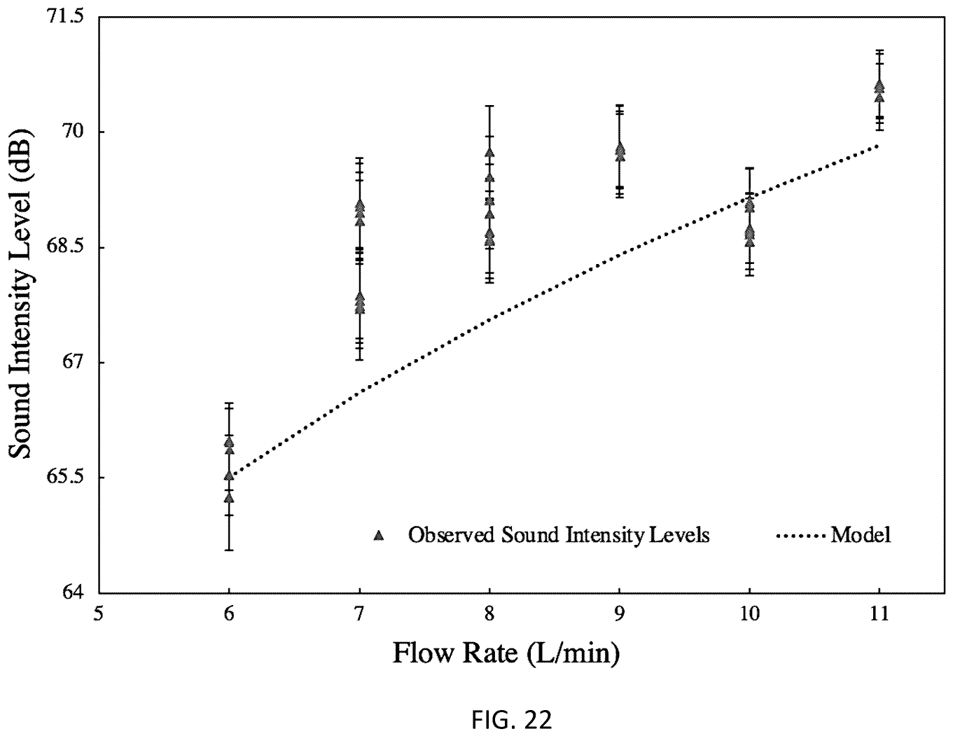

The next plot examined was sound intensity level as a function of flow rate for the second straw size, as shown in FIG. 22. The observed flow rates at 6 and 10 L/min closely match the model; on the other hand, the flow rates at 7, 8, and 9 L/min deviate from the model at a range from 1.4 to 1.8 dB. This variation likely could have been due to some error when performing the experiment.

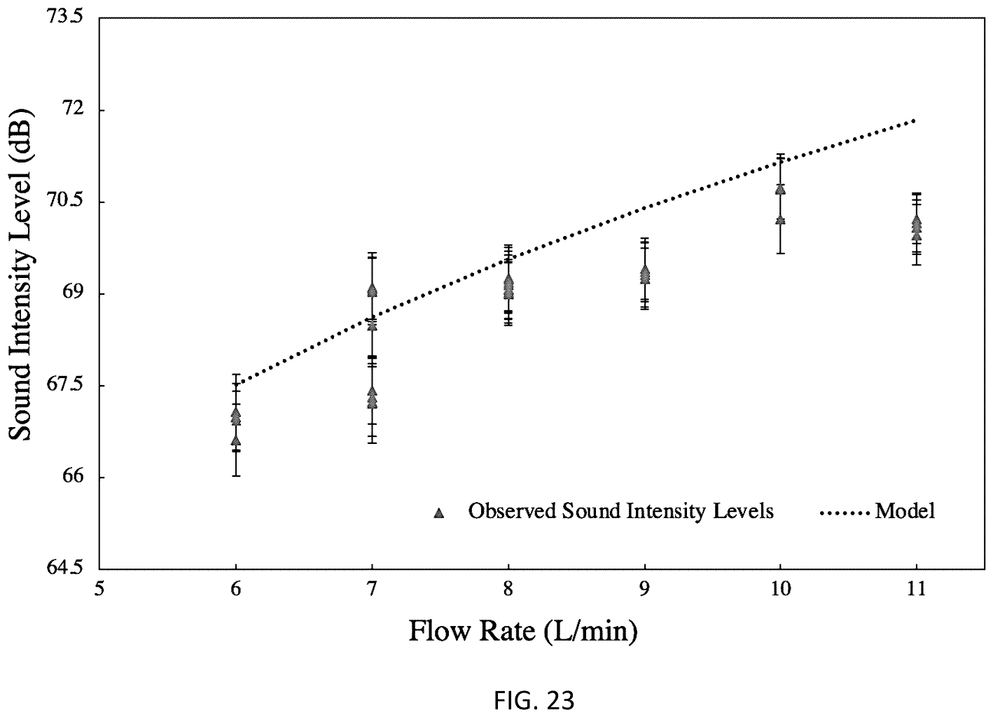

The last straw size is plotted in FIG. 23. It is expected there would be a trend of increasing sound intensity levels with increasing flow rate, however, there appears to be a trend of decreasing sound intensity levels with increasing flow rate. The 11 L/min flow rate held the largest deviation at 1.7 dB. One possible source for the error is that since this was the largest straw tested, it was not a perfect fit around the tube it was connected to. With increasing flow, there was most likely some air escaping due to imperfect sealing between the straw and the tube. This would make sense because the sound levels begin to decrease with increasing air flow, indicating more air was escaping. For future testing, it's recommended to ensure a proper seal by using a larger tube to connect the straw to. This would help with the accuracy and consistency of sound intensity measurements.

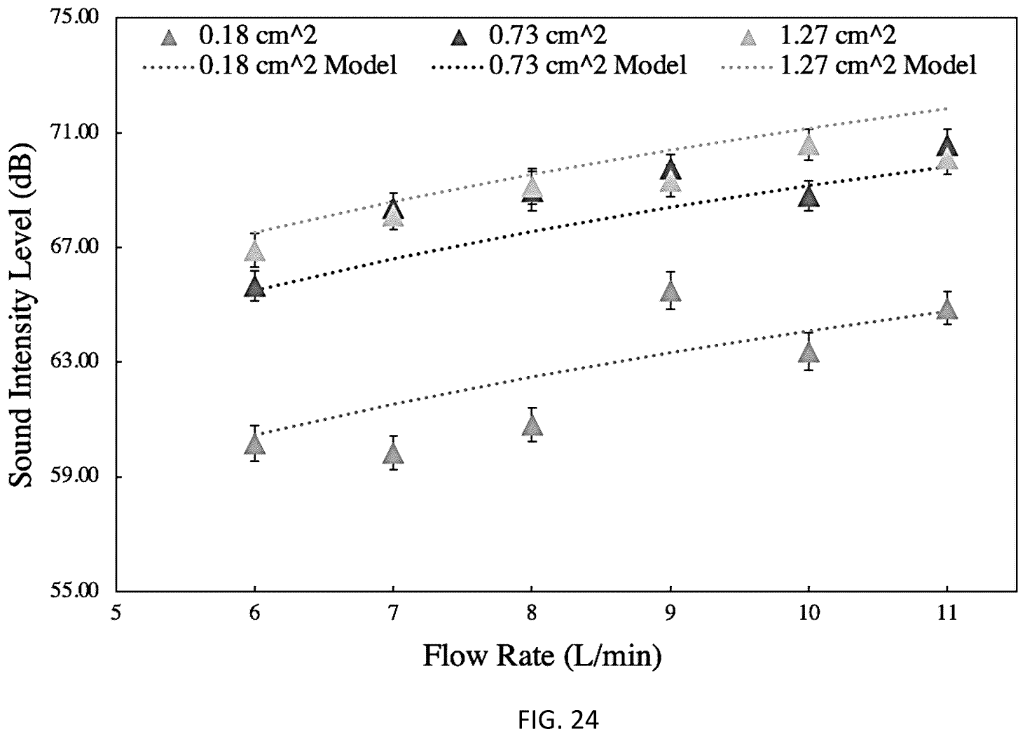

For all three straws tested, all the data points were averaged out at each flow rate and were plotted on the same graph, as shown in FIG. 24. For the cross-sectional area of 0.18 cm², the sound intensity levels ranged from 59.84 dB to 65.50 dB. For the cross-sectional area of 0.73 cm², the sound intensity levels ranged from 65.66 dB to 70.57 dB. For the cross-sectional area of 1.27 cm², the sound intensity levels ranged from 66.90 dB to 70.59 dB. As expected, as the cross-sectional area increases, the sound intensity levels generally increase. Comparing the cross-sectional areas at 0.73 cm² and 1.27 cm², the sound intensity levels intersect, indicating there is not a clear distinction between them. This is most likely because there wasn't a large enough size difference between those two straws. The difference in straw diameters between the smallest straw and middle straw was 0.2 cm, while the difference between the middle straw size and largest straw size was only 0.12 cm. This size difference of 0.12 cm was most likely not significant enough to see an increase in sound levels. It is important for future testing to conduct more runs at different straw diameters to see clearer distinctions.

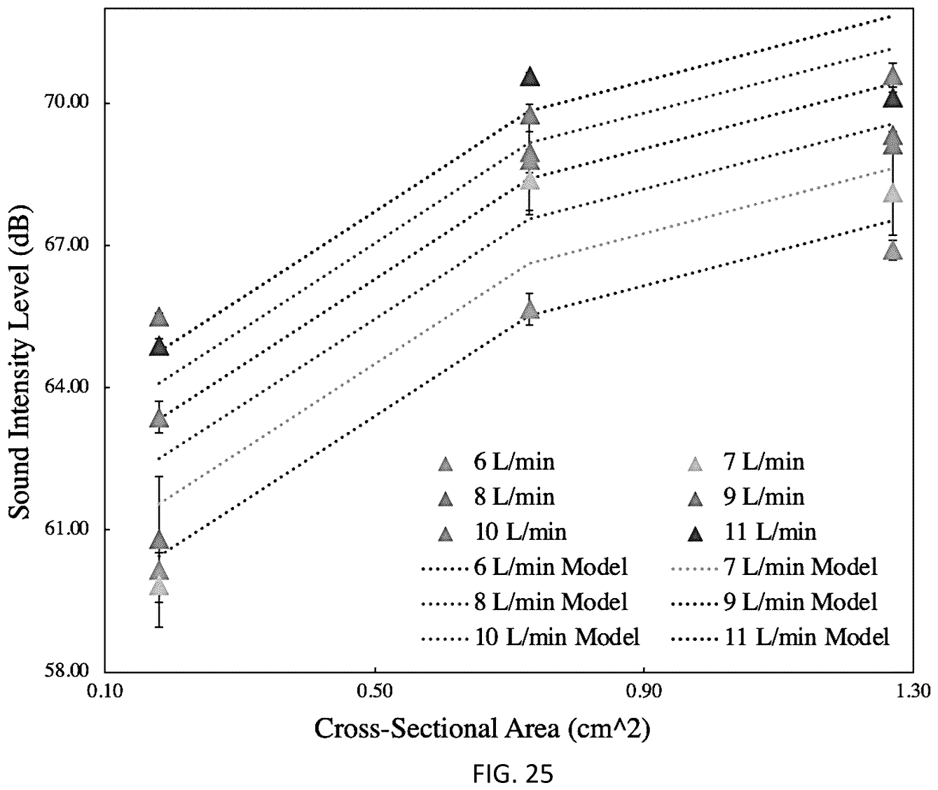

As mentioned previously, it is expected that with increasing cross-sectional area, the sound intensity levels will increase as well. This is largely because more volume can flow through the tube, allowing for more air molecule collisions, creating turbulence. In the airways, more interaction with the irregular surfaces should lead to more intense sound intensity levels; as a result, this same result should be expected with the simulated straws used. FIG. 25 shows the results of the sound intensity levels as a function of cross-sectional area. The figure also plots all the flow rates tested. Generally, as cross-sectional area increases, the sound intensity levels also increase but it is not a perfect trend. There are variations and deviations away from the model. Across all cross-sectional areas and flow rates, the observed values deviated at an average of about 1 dB away from the model. At the cross-sectional area of 0.73 cm², observed sound intensity levels are greater than the predicted with an average deviation of 0.96 dB from the model. At the cross-sectional area of 1.27 cm², observed sound intensity levels are lower than the predicted, with an average deviation of 0.82 dB from the model. The largest deviations occur at the smallest cross-sectional area of 0.18 cm², with an average of 1 dB. It was expected, however, that the smallest cross-sectional area would produce the least variation as it is most susceptible to laminar flow, which would best yield consistent and accurate results in comparison to turbulent flow.

In conclusion, as cross-sectional area and flow rate increase, sound intensity level generally increases but is not linear. There were also some deviations away from the model when comparing the observed values to the recorded values. This variability in results could be due to tests being conducted on different days, where the background noises could have varied, yielding inconsistent results. In addition, as a larger air flow rate was supplied, the pump generated more noise as a result. This also could have had an impact on the sound levels recorded by the meter.

Some future recommendations include 3D-printing vessels that better simulate the vessels within the respiratory system. The silicone straws used are not an accurate representation of the inner surfaces of tubes, especially for an individual with respiratory illnesses who could have irregularities within the inner surfaces of the tube walls due to airway obstruction. It is also recommended to test more cross-sectional areas to have a wider range of data to analyze. Lastly, since the pump generates significant noise that can impact the recorded sound levels, it is recommended to use a gas cylinder and supply air through a direct line.

Nonetheless, the measurement of sound intensity levels can serve as an important indicator in assessing the airways. By comparing measured sound intensity levels to established norms, deviations can be identified that may indicate airway obstruction or reduced airflow. These findings can then be correlated to their respective respiratory illnesses. This research ultimately provides an alternative approach for diagnosing respiratory conditions.

### Computing Environment

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C #, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

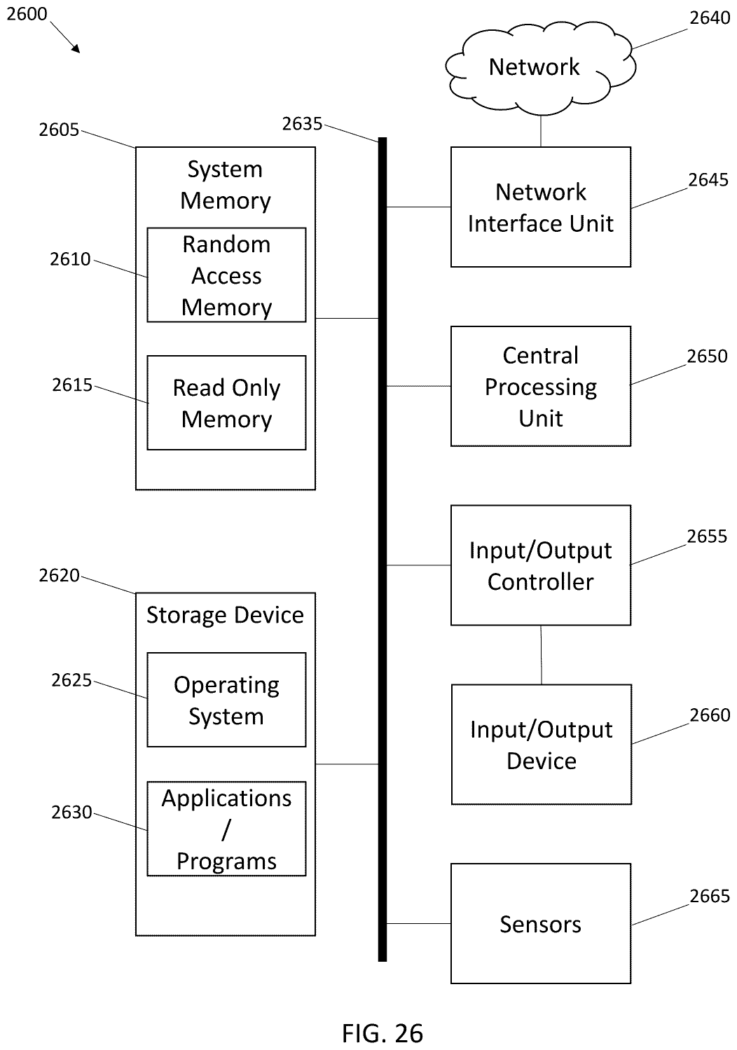

FIG. 26 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 26 depicts an illustrative computer architecture for a computer 2600 for practicing the various embodiments of the invention. The computer architecture shown in FIG. 26 illustrates a conventional personal computer, including a central processing unit 2650 (“CPU”), a system memory 2605, including a random-access memory 2610 (“RAM”) and a read-only memory (“ROM”) 2615, and a system bus 2635 that couples the system memory 2605 to the CPU 2650. A basic input/output system containing the basic routines that help to transfer information between elements within the computer, such as during startup, is stored in the ROM 2615. The computer 2600 further includes a storage device 2620 for storing an operating system 2625, application/program 2630, and data.

The storage device 2620 is connected to the CPU 2650 through a storage controller (not shown) connected to the bus 2635. The storage device 2620 and its associated computer-readable media, provide non-volatile storage for the computer 2600. Although the description of computer-readable media contained herein refers to a storage device, such as a hard disk or CD-ROM drive, it should be appreciated by those skilled in the art that computer-readable media can be any available media that can be accessed by the computer 2600.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information, and which can be accessed by the computer.

According to various embodiments of the invention, the computer 2600 may operate in a networked environment using logical connections to remote computers through a network 2640, such as TCP/IP network such as the Internet or an intranet. The computer 2600 may connect to the network 2640 through a network interface unit 2645 connected to the bus 2635. It should be appreciated that the network interface unit 2645 may also be utilized to connect to other types of networks and remote computer systems.

The computer 2600 may also include an input/output controller 2655 for receiving and processing input from a number of input/output devices 2660, including a keyboard, a mouse, a touchscreen, a camera, a microphone, a controller, a joystick, or other type of input device. Similarly, the input/output controller 2655 may provide output to a display screen, a printer, a speaker, or other type of output device. The computer 2600 can connect to the input/output device 2660 via a wired connection including, but not limited to, fiber optic, ethernet, or copper wire or wireless means including, but not limited to, Bluetooth, Near-Field Communication (NFC), infrared, or other suitable wired or wireless connections.

As mentioned briefly above, a number of program modules and data files may be stored in the storage device 2620 and RAM 2610 of the computer 2600, including an operating system 2625 suitable for controlling the operation of a networked computer. The storage device 2620 and RAM 2610 may also store one or more applications/programs 2630. In particular, the storage device 2620 and RAM 2610 may store an application/program 2630 for providing a variety of functionalities to a user. For instance, the application/program 2630 may comprise many types of programs such as a word processing application, a spreadsheet application, a desktop publishing application, a database application, a gaming application, internet browsing application, electronic mail application, messaging application, and the like. According to an embodiment of the present invention, the application/program 2630 comprises a multiple functionality software application for providing word processing functionality, slide presentation functionality, spreadsheet functionality, database functionality and the like.

The computer 2600 in some embodiments can include a variety of sensors 2665 for monitoring the environment surrounding and the environment internal to the computer 2600. These sensors 2665 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

The following publications are each hereby incorporated herein by reference in their entirety:
1.) Respiratory system: Functions, facts, Organs & Anatomy. Cleveland Clinic. (n.d.). Retrieved Apr. 24, 2022, from https://my.clevelandclinic.orehealth/articles/21205-respiratory-system#:˜:text=The%20respiratory%20system%20is%20the,waste%20gases%20like%20carbon %20dioxide
2.) Betts, J. G., Desaix, P., Johnson, E., Johnson, J. E., Korol, O., Kruse, D., Poe, B., Wise, J., Womble, M. D., & Young, K. A. (2017). Anatomy & Physiology. OpenStax College, Rice University.
3.) Hallett, S., Toro, F., & Ashurst, J. V. (2021). Physiology, Tidal Volume. In StatPearls. StatPearls Publishing.
4.) Do, Q. T., Lipatov, K., Wang, H.-Y., Pickering, B. W., & Herasevich, V. (2021). Classification of respiratory conditions using auscultation sound. 2021 43rd Annual International Conference of the IEEE Engineering in Medicine & Biology Society (EMBC). https://doi.org/10.1109/embc46164.2021.9630294
5.) Respiratory Therapy Zone. (2022, March 3). What are the parts of a stethoscope?. Respiratory Therapy Zone. Retrieved Apr. 24, 2022, from https://www.respiratorytherapyzone.com/parts-of-a-stethoscope/6.)
6.) Zimmerman, B., & Williams, D. (2021). Lung Sounds. In StatPearls. StatPearls Publishing.
7.) Respiratory Therapy Zone. (2022, April 21). Lung sounds: The ultimate guide to breath sounds and auscultation. Respiratory Therapy Zone. Retrieved Apr. 24, 2022, from https://www.respiratorytherapyzone.com/breath-sounds-guide/8.)
8.) American Thoracic Society and others. (1977). Updated Nomenclature for Membership Reaction. Reports of the ATS-ACCP Ad Hoc Committee, 3.
9.) Bohadana, A., lzbicki, G., & Kraman, S. S. (2014). Fundamentals of Lung Auscultation. New England Journal of Medicine, 370(8), 744-751. https://doi.org/10.1056/NEJMra1302901
10.) Chamberlain, N. (2014). Respiratory Airway Infections. https://www.atsu.edu/faculty/chamberlain/website/lectures/lecture/reairin2.htm
11.) Forgacs, P. (1978). The Functional Basis of Pulmonary Sounds. Applied Cardiopulmonary Physiology, 73(3), 399-405. https://doi.org/10.1378/chest.73.3.399
12.) Hsu, F.-S., Huang, S.-R., Huang, C.-W., Huang, C.-J., Cheng, Y.-R., Chen, C.-C., Hsiao, J., Chen, C.-W., Chen, L.-C., Lai, Y.-C., Hsu, B.-F., Lin, N.-J., Tsai, W.-L., Wu, Y.-L., Tseng, T.-L., Tseng, C.-T., Chen, Y.-T., & Lai, F. (2021). Benchmarking of eight recurrent neural network variants for breath phase and adventitious sound detection on a self-developed open-access lung sound database—HF_Lung_V1. PLOS ONE, 16(7), e0254134. https://doi.org/10.1371/journal.pone.0254134
13.) Meslier, N., Charbonneau, G., & Racineux, J. (1995). Wheezes. European Respiratory Journal, 8(11), 1942.
14.) Munakata, M., Ukita, H., Doi, I., Ohtsuka, Y., Masaki, Y., Homma, Y., & Kawakami, Y. (1991). Spectral and waveform characteristics of fine and coarse crackles. Thorax, 46(9), 651. https://doi.org/10.1136/thx.46.9.651
15.) Nagasaka, Y. (2012). Lung Sounds in Bronchial Asthma. Allergology International, 61(3), 353-363. https://doi.org/10.2332/allergolint.12-RAI-0449
16.) Pasterkamp, H., Kraman, S. S., & Wodicka, G. (1997). Respiratory Sounds: Advances Beyond the Stethescope. Am J Respir Crit Care Med, 156(3), 974-987. http://doi.org/10.1056/NEJMra1302901
17.) Pramono, R. X. A., Bowyer, S., & Rodriguez-Villegas, E. (2017). Automatic adventitious respiratory sound analysis: A systematic review. PLoS ONE, 12(5). Scopus. https://doi.org/10.1371/journal.pone.0177926
18.) Sarkar, M., Madabhavi, I., Niranjan, N., & Dogra, M. (2015). Auscultation of the respiratory system. Annals of Thoracic Medicine, 10(3), 158-168. Scopus. https://doi.org/10.4103/1817-1737.160831
19.) Mayo Foundation for Medical Education and Research. (2022, March 5). Asthma. Mayo Clinic. Retrieved Apr. 24, 2022, from https://www.mayoclinic.org/diseases-conditions/asthma/symptoms-causes/syc-20369653
20.) Mayo Foundation for Medical Education and Research. (2020, April 15). COPD. Mayo Clinic. Retrieved Apr. 24, 2022, from https://www.mayoclinic.org/diseases-conditions/copd/symptoms-causes/syc-20353679
21.) Mayo Foundation for Medical Education and Research. (2017, April 11). Bronchitis. Mayo Clinic. Retrieved Apr. 24, 2022, from https://www.mayoclinic.org/diseases-conditions/bronchitis/symptoms-causes/syc-20355566
22.) Mayo Foundation for Medical Education and Research. (2020, June 13). Pneumonia. Mayo Clinic. Retrieved Apr. 24, 2022, from https://www.mayoclinic.org/diseases-conditions/pneumonia/symptoms-causes/syc-20354204
23.) Safshekan, F., Tafazzoli-Shadpour, M., Abdouss, M., Behgam Shadmehr, M., & Ghorbani, F. (2017). Investigation of the Mechanical Properties of the Human Tracheal Cartilage. Tanaffos, 16(2), 107-114.
24.) Safshekan, F., Tafazzoli-Shadpour, M., Abdouss, M., & Shadmehr, M. (2016). Mechanical characterization and constitutive modeling of human trachea: Age and gender dependency. Materials, 9(6), 456. https://doi.org/10.3390/ma9060456
25.) Breatnach, E., Abbott, G. C., & Fraser, R. G. (1984). Dimensions of the normal human trachea. AJR. American journal of roentgenology, 142(5), 903-906. https://doi.org/10.2214/ajr.142.5.903
26.) Wang, J.-Y., Mesquida, P., Pallai, P., Corrigan, C. J., & Lee, T. H. (2011, November 23). Dynamic Elastic Properties of Human Bronchial Airway Tissues. arXiv.org. Retrieved Apr. 24, 2022, from https://arxiv.org/abs/1111.5645
27.) Lee, J. W., Son, J. S., Choi, J. W., Han, Y. J., & Lee, J. R. (2014). The comparison of the lengths and diameters of main bronchi measured from two-dimensional and three-dimensional images in the same patients. Korean journal of anesthesiology, 66(3), 189-194. https://doi.org/10.4097/kjae.2014.66.3.189
28.) Enna, S. J., & Bylund, D. B. (2011). The Comprehensive Pharmacology Reference. XPharm.
29.) Overview of materials for Silicone Rubber. MatWeb. (2022). Retrieved Apr. 24, 2022, from https://www.matweb.com/search/DataSheet.aspx?MatGUID=cbe7a469897a47eda563816c86a 73520&ckck=1
30.) Properties: Silicone rubber. AZoM.com. (2022, April 13). Retrieved Apr. 24, 2022, from https://www.azom.com/properties.aspx?ArticleID=920
31.) GBD Chronic Respiratory Disease Collaborators. Prevalence and attributable health burden of chronic respiratory diseases, 1990-2017: a systematic analysis for the Global Burden of Disease Study 2017. Lancet Respir Med. 2020 June; 8(6):585-596. doi:10.1016/52213-2600(20)30105-3. PMID: 32526187; PMCID: PMC7284317.
32.) Mukherjee H, Sreerama P, Dhar A, Obaidullah S M, Roy K, Mahmud M, Santosh K C. Automatic Lung Health Screening Using Respiratory Sounds. J Med Syst. 2021 Jan. 11; 45(2):19. doi: 10.1007/s10916-020-01681-9. PMID: 33426615; PMCID: PMC7797201
33.) Gurung A, Scrafford C G, Tielsch J M, Levine O S, Checkley W. Computerized lung sound analysis as diagnostic aid for the detection of abnormal lung sounds: a systematic review and meta-analysis. Respir Med. 2011 September; 105(9):1396-403. doi: 10.1016/j.rmed.2011.05.007. Epub 2011 Jun. 14. PMID: 21676606; PMCID: PMC3227538
34.) “The Respiratory System.” The Lung Association, n.d., https://www.lung.ca/lung-health/lung-info/respiratory-system
35.) Pleil J D, Ariel Geer Wallace M, Davis M D, Matty C M. The physics of human breathing: flow, timing, volume, and pressure parameters for normal, on-demand, and ventilator respiration. J Breath Res. 2021 Sep. 27; 15(4):10.1088/1752-7163/ac2589. doi: 10.1088/1752-7163/ac2589 PMID: 34507310; PMCID: PMC8672270
36.) The Mechanics of Human Breathing I Biology for Majors I I. (n.d.). Lumen Learning. Retrieved Apr. 28, 2023, from https://courses.lumenlearning.com/wm-biology2/chapter/the-mechanics-of-human-breathing/9. Hallett S, Toro F, Ashurst J V. Physiology, Tidal Volume. [Updated 2022 May 8]. In: StatPearls [Internet]. Treasure Island (FL): StatPearls Publishing; 2023 Jan-. Available from: https://www.ncbi.nlm.nih.gov/books/NBK482502/37.)
37.) Verywell Health. (2021). Understanding Normal and Abnormal Breath Sounds. Retrieved from https://www.verywellhealth.com/breath-sounds-4686352 #toc-normal-breath-sounds
38.) Baňačka, R. Auscultation of Lung Sounds and Murmurs [Online]. Available: http://patfyz.medic.upjs.sk/simulatorvzorky/Respiratory%20auscultation.htm [Accessed on Apr. 22, 2023].
39.) 17. Loring, S. H., Garcia-Jacques, M., & Malhotra, A. (2009). Pulmonary characteristics in COPD and mechanisms of increased work of breathing. Journal of applied physiology (Bethesda, Md.: 1985), 107(1), 309-314. https://doi.org/10.1152/japplphysio1.00008.2009
40.) Gavriely N, Cugell D W. Airflow effects on amplitude and spectral content of normal breath sounds. J Appl Physiol (1985). 1996 January; 80(1):5-13. doi: 10.1152/japp1.1996.80.1.5. PMID: 8847331.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. An auscultation device, comprising:
a wearable apparatus positionable proximate to a lung;
a plurality of audio sensors embedded in the wearable apparatus configured to record lung sounds, wherein the plurality of audio sensors are positioned at locations of the wearable apparatus corresponding to lung auscultation points;
a microcontroller embedded in the wearable apparatus configured to control operation of the plurality of audio sensors;
a transceiver embedded in the wearable apparatus; and
a power source embedded in the wearable apparatus.

2. The device of claim 1, wherein the wearable apparatus comprises a vest, a strap, a jacket, or a shirt.

3. The device of claim 1, wherein the lung auscultation points include at least one of an apex of a lung, an upper lobe of the lung, a middle lobe of the lung, and a lower lobe of the lung.

4. The device of claim 1, wherein the wearable apparatus is configured to accommodate variations in body weight or size.

5. The device of claim 1, wherein the wearable apparatus is configured to accommodate male and female patients.

6. The device of claim 1, wherein the audio sensor comprises a diaphragm, a chest piece, and a sound meter.

7. The device of claim 1, wherein the audio sensor comprises a stethoscope.

8. The device of claim 1, wherein the power source comprises a battery.

9. The device of claim 1, wherein the plurality of audio sensors include four anterior sensors and eight posterior sensors.

10. An auscultation system, comprising:
an auscultation device comprising a wearable apparatus positionable proximate to a lung, a plurality of audio sensors embedded in the wearable apparatus configured to record lung sounds, wherein the plurality of audio sensors are positioned at locations of the wearable apparatus corresponding to lung auscultation points, a microcontroller embedded in the wearable apparatus configured to control operation of the plurality of audio sensors, a transceiver embedded in the wearable apparatus, and a power source embedded in the wearable apparatus; and
a computing system communicatively connected to the auscultation device, comprising a processor and a non-transitory computer-readable medium with instructions stored thereon, which when executed by a processor, perform steps comprising:
establishing a baseline lung sound spectrum via data received from the auscultation device;
recording a lung sound spectrum via data received from the auscultation device; and
identifying a change in a patient's lung sounds over time via comparing the recorded lung sound spectrum to the baseline lung sound spectrum.

11. The system of claim 10, wherein the auscultation device is wirelessly communicatively connected to the computing system via a wireless communication protocol comprising 3G, 4G/LTE, 5G, Bluetooth, Bluetooth Low Energy (BLE), Zigbee, near-field communication (NFC), or infrared.

12. The system of claim 10, further comprising a network communicatively connected to the auscultation device and computing system.

13. The system of claim 12, wherein the auscultation device and computing system are wirelessly communicatively connected to the network via a wireless communication protocol comprising 3G, 4G/LTE, 5G, Bluetooth, Bluetooth Low Energy (BLE), Zigbee, near-field communication (NFC), or infrared.

14. A method for monitoring a patient's change in lung sounds over time, comprising:
providing the system of claim 10 proximate to a patient's lung;
establishing a baseline lung sound spectrum via the auscultation device;
recording a lung sound spectrum via the auscultation device; and
identifying a change in a patient's lung sounds over time via comparing the recorded lung sound spectrum to the baseline lung sound spectrum.

15. The method of claim 14, further comprising:
establishing a background noise spectrum;
calculating a background subtracted lung sound spectrum and baseline lung sound spectrum by subtracting the background noise spectrum from each respective lung sound spectrum.

16. The method of claim 14, wherein the step of recording a lung sound spectrum is performed continuously or periodically.

17. The method of claim 14, further comprising:
determining at least one of a treatment effectiveness, a disease state, and a disease progression based on the change in a patient's lung sounds over time; and
providing at least one of the treatment effectiveness, the disease state, and the disease progression to a practitioner.

18. The method of claim 17, wherein the at least one of the treatment effectiveness, the disease state, and the disease progression is provided to the practitioner located remotely.

19. The method of claim 17, wherein the at least one of the treatment effectiveness, the disease state, and the disease progression is provided to the practitioner in real time.

20. The method of claim 14, wherein the change in a patient's lung sounds over time is identified via a machine learning algorithm.
